(19) United States
(12) Patent Application Publication
(10) Pub. No.: US 2006/0246544 A1
(43) Pub. Date: Nov. 2, 2006

(54) Manufacturing process for the production of peptides grown in insect cell lines

(75) Inventors: Yun Kang, Livingston, NJ (US); Walter Scott Willett, Doylestown, PA (US); Thomas J. Klimek, Philadelphia, PA (US); Basil Amir Campbell, Gaithersburg, MD (US); Paul M. Cino, Jamison, PA (US); Bradley Thomas, Quakertown, PA (US); John V. Bermel, Telford, PA (US); Chun-Chiang Chen, Gaithersburg, MD (US)

Correspondence Address: MORGAN, LEWIS & BOCKIUS LLP (SF), 2 PALO ALTO SQUARE, 3000 El Camino Real, Suite 700, PALO ALTO, CA, 94306, US

(73) Assignee: Neose Technologies,Inc., Horsham, PA (US)

(21) Appl. No.: 11/396,215

(22) Filed: Mar. 30, 2006

Related U.S. Application Data
(60) Provisional application No. 60/666,545, filed on Mar. 30, 2005.
(60) Provisional application No. 60/678,822, filed on May 6, 2005.
(60) Provisional application No. 60/729,240, filed on Oct. 19, 2005.

(51) Int. Cl. C12P 21/06; C12N 9/24; C12N 5/06; C07K 14/53; C07K 14/51

(52) U.S. Cl. 435/69.1; 435/320.1; 435/348; 435/200; 530/351; 530/414; 530/399

(57) ABSTRACT

The present invention provides a manufacturing method for the production of peptides that are grown in insect cell lines. The peptides are grown in insect cell cultures that are infected with baculovirus particles in a culture supplemented with a lipid mixture. The peptides are then isolated from the insect cell culture using a method that employs a tangential flow filtration cascade. The isolated peptides are glycopeptides having an insect specific glycosylation pattern. The glycopeptides may then be conjugated to a modifying group via linkage through a glycosyl linking group interposed between and covalently attached to the peptide and the modifying group. The conjugates are formed from glycosylated peptides by the action of a glycosyltransferase.

### CROSS-REFERENCES TO RELATED APPLICATIONS

The present application claims priority to U.S. Provisional Patent Application No. 60/678,822, filed May 6, 2005; U.S. Provisional Patent Application No. 60/729,240, filed Oct. 19, 2005; and U.S. Provisional Patent Application No. 60/666,545, filed Mar. 30, 2005 each of which is incorporated herein by reference in its entirety for all purposes.

### FIELD OF THE INVENTION

The invention pertains to the field of peptide manufacturing. In particular, the invention pertains to a production method for manufacturing glycosylated peptides using a baculovirus expression vector system.

### BACKGROUND OF THE INVENTION

With the development and refinement of recombinant-DNA techniques, it was anticipated that large-scale production of therapeutically valuable peptides could be achieved in a cost effective manner using genetically modified bacteria. This expectation has to some extent been borne out as recombinant bacteria are an important source for the production of many biological products, including therapeutic peptides. Unfortunately however, many heterologous proteins produced in E. coli are insoluble and difficult to purify. Furthermore, the majority of commercially attractive proteins require post-translational modifications, such as glycosylation, before they can become biologically active proteins, and bacterial cells cannot make these post-translational modifications.

It is well known in the art that proper glycosylation is a critically important factor influencing the in vivo half life and immunogenicity of therapeutic peptides. Indeed, humans will typically tolerate only those biotherapeutics that have particular types of carbohydrate attachments and will often reject glycoproteins that include non-mammalian oligosaccharide attachments. For instance, poorly glycosylated peptides are recognized by the liver as being “old” and thus, are more quickly eliminated from the body than are properly glycosylated peptides. In contrast, hyperglycosylated peptides or incorrectly glycosylated peptides can be immunogenic. Because of the requirement for post-translational modifications, particularly the requirement for proper glycosylation, mammalian cells are often the cell type of choice for the production of recombinant therapeutic glycoproteins.

Since all mammals produce glycans of similar structure, Chinese Hamster Ovary (CHO), Baby Hamster Kidney (BHK), and Human Embryonic Kidney-293 (HEK-293) are often the preferred host cells for production of glycoprotein therapeutics. Unfortunately however, mammalian cell cultures are characterized by low cell densities and low growth rates. Furthermore, maintenance and growing of mammalian cell cultures can be very expensive, gene manipulations are difficult, and mammalian cells potentially contain oncogenes or viral DNA that can affect human subjects. Therefore, recombinant protein products produced in mammalian cells require extensive testing for safety.

To overcome the problems associated with peptide production in mammalian cell cultures, insect cell culture systems have been developed. Insect cells recognize the signal sequences and possess the metabolic pathways for processing glycoproteins in a manner similar to mammalian cells. Thus, there has been a great deal of interest in using insect cells in combination with the baculovirus expression system for the production of recombinant glycoproteins, and so far, hundreds of proteins have been expressed in insect cell cultures with the baculovirus expression vector system (BEVS).

The baculovirus expression vector system employs insect cells that are derived from lepidopteran larvae (referred to herein as insect cells). The BEVS has several advantages as a recombinant protein production system. For example, the time from gene isolation to BEVS expression can be as short as 4-6 weeks. Production levels are typically higher than those achievable using mammalian cell lines, and adventitious viruses (commonly found in mammalian tissue culture cells) are typically absent. Importantly, as noted above, insect cells are able to recognize the co- and post-translational signals of higher eukaryotes, resulting in processing such as phosphorylation, proteolytic processing, carboxyl methylation, and glycosylation.

Given the many advantages of the BEVS over mammalian expression systems for the production of recombinant glycoproteins, it is not surprising that interest in improving insect cell culture technology has increased in recent years (see e.g., Schlaeger, E. (1996) Cytotechnology 20:57-70, for a review).

In general, the principles that apply to the growth of insect cell cultures are the same as those for mammalian cell culture, with the major difference being the nature of the growth medium used. For example, compared to mammalian cell culture media, insect cell culture media used in large-scale production processes typically contain significantly increased concentrations of many amino acids, vitamins, and salts, and the media is also more acidic.

Insect cells can easily be grown in shaker flasks. However, cell growth and recombinant protein production with BEVS on a large scale can be difficult. For instancce, because insect cells require 3-10 fold higher oxygen concentrations than mammalian cells, the cultures must be sparged with air to supply the necessary oxygen. However, insect cells are shear-sensitive due to their large size and lack of a cell wall. Virus-infected insect cells are even more shear-sensitive, since they swell to twice their original size upon virus infection. Thus, the cells must be protected from shear by air bubbles in gas sparged bioreactors. To protect the cells from shear stress a block co-polymer surfactant, such as Pluronic F-68, is added to large-scale cultures.

Insect cell cultures also require supplementation with serum (e.g., fetal bovine serum). The serum provides growth promoting hormones e.g., sterols, as well as lipids, including both essential and non-essential fatty acids, and other low molecular weight substances required for insect cell growth. Unfortunately, in addition to the batch to batch variation in the quality of serum, serum also has the potential for contamination with adventitious agents and mycoplasma, and is very expensive. Indeed, sometimes the cost of the serum accounts for more than 50% of the total medium cost. Furthermore, serum proteins can hinder the downsteam processing of therapeutic peptides and proteins under production.

Because of the many drawbacks associated with serum use on a large-scale, cost-effective substitutes for serum were developed for large-scale production processes. Those substitutes include free medium compositions containing protein hydrolysates and lipids.

Lipids can be used to meet the requirement of insect cells for certain sterols and essential and non-essential fatty acids, and thus can supply many of the components necessary for the growth of insect cell cultures. A lipid formulation particularly favored in the art for the supplementation of insect cell cultures is disclosed by Inlow et al., (1989) J. Tissue Culture Meth. 12:13-16; and is shown in Table 1 below.

TABLE 1
Lipid Composition for 100 Liters Culture from Inlow et al., (1989) supra

| Components | Amount (mg) |
|---|---|
| Cholesterol | 450 |
| Tween 80 | 2400 |
| Cod Liver Oil | 1000 |
| d-Tocopherol Acetate | 200 |

This formulation has been used advantageously by numerous investigators and manufacturers to enhance the productivity and growth parameters of insect cell cultures (see e.g., Schlaeger, E. (1996) Cytotechnology 20:57-70, for a review).

Typically, it is expected that an increase in cell growth correlates with increased productivity. However, Schlaeger (supra) reports that improvements that lead to increases in cell density do not necessarily correlate with increased yields of recombinant protein and extracellular baculoviruses. Therefore, Schlaeger concludes that a culture medium optimized for cell growth and density does not necessarily fulfill all the requirements for an optimal peptide/protein production process.

Consistent with the conclusion of Schlaeger, Gilbert et al. (1996) Cytotechnology 22:211-216, found that in order to achieve efficient infection and protein production, lipids were required in the cellular growth phase that precedes infection with baculovirus. Gilbert et al. also found that the presence or absence of lipids during or immediately after infection had no effect on the expression of proteins from the infecting baculovirus.

To test the requirement for lipids in insect cell culture, Gilbert et al. grew two seed cultures; one with lipid supplementation, and the other without lipid supplementation. When the cells were at a density sufficient for infection, the seed cultures were split and centrifuged, and after centrifugation the split cultures were resuspended. One half of the culture was resuspended in media containing lipids, and the other half of the culture was resuspended in media devoid of lipids. Infection with baculovirus comprising a recombinant β-gal reporter gene was carried out for two hours. Following the infection period, the cultures were again split and centrifuged, and the split cultures resuspended in media either containing or devoid of lipids. The cultures were grown and the reporter gene was expressed for 72-96 hours.

From these experiments, Gilbert et al. concluded that the protein expression level as evidenced by the amount of β-gal activity in the culture, was influenced by whether or not the original seed culture included lipids, and not by whether lipids were present in the culture at the time of infection.

The development of insect cell culture media for large-scale protein production is still in need of improvements that will boost the productivity of the cell cultures beyond a level, which follows from improvements in cell growth parameters. In addition, peptide purification processes are needed that are efficient in removing a variety of contaminants, such as cellular proteins and potential pathogenic viruses, thereby providing high quality recombinant peptides, which are safe for use in humans. As will be clear from the disclosure that follows, the present invention meets this, and other needs.

### SUMMARY OF THE INVENTION

The present invention provides methods for the large-scale production of peptides and glycopeptides. In one aspect the invention provides a method of generating cell cultures that contain a recombinant peptide in high concentration and improved purity. In another aspect, the invention provides novel methods of purifying a recombinant peptide. Combined, these methods form an efficient and cost-effective peptide production process that can provide high-quality recombinant peptides. In some embodiments, the recombinant peptides so produced are glycopeptides and are further processed to elaborate the structure of their glycosyl residues. In other embodiments the glycopeptides are used to create a glycopeptide conjugate, e.g., a conjugate between a peptide (glycopeptide) and a polymer (e.g, polyethylene glycol).

The invention includes a newly discovered infection procedure that provides cell cultures containing a recombinant peptide in unexpectedly high concentration and purity. The present inventors have discovered that, contrary to the teachings of the prior art, infecting insect cells with a recombinant baculovirus when a lipid mixture is present in the cell culture at the time of infection, increases the amount of peptide expressed by the insect cells. In some embodiments, the amount of peptide in the cell culture is increased by about 82% when compared to the amount in a culture not supplemented with the lipid mixture. In other embodiments the amount of recombinant peptide in the cell culture is increased by about 38% when compared to the amount in a culture supplemented with a commercial lipid mixture. The method is particularly useful for large-scale production of glycopeptides.

An exemplary method of the invention, includes infecting insect cells in an insect cell culture with a recombinant baculovirus that includes a nucleotide sequence encoding a peptide. The infecting takes place in an insect cell culture that is supplemented with a lipid mixture. The infected insect cells are grown to produce the peptide encoded by the nucleic acid sequence. The peptide so produced has an insect-specific glycosylation pattern. In one embodiment, the peptide so produced has a substantially uniform, insect-specific glycosylation pattern.

The invention also includes methods of purifying a recombinant peptide. In one aspect, the invention provides a method of purifying a recombinant peptide using a “tangential flow filtration (TFF) cascade”. This conditioning step is preferably performed prior to chromatographic purification and delivers the peptide in a concentration and purity that allows subsequent purification steps to be more efficient and increases the recovery of peptide from certain purification steps.

In another aspect, the invention includes a novel method of inactivating viral particles in a mixture containing a recombinant peptide. In one embodiment the viral inactivation method includes lowering the pH of a peptide solution to a value suitable to decrease the viability of certain viruses (e.g non-enveloped viruses) and maintaining this low pH (e.g. pH about 2.2) for a suitable amount of time (e.g. about 1 hour), before the pH is raised. The pH value and the holding period are selected to minimize degradation of the peptide while exposing the peptide to the low-pH. In some embodiments, the purified peptide is surprisingly stable at the selected low pH.

In a further aspect, the invention provides a method of removing a low-molecular weight impurity from a peptide solution by hydrophobic interaction chromatography. Certain impurities (e.g. low-molecular weight cellular proteins) are released into the cell culture medium during expression of the peptide (e.g by a baculovirus expression vector system). In some embodiments, those contaminants are difficult to separate from the purified peptide. The present invention provides methods of separating the recombinant peptide of interest from a low-molecular weight impurity. This method produces a peptide that is unexpectedly pure.

In another aspect, the invention provides methods of increasing the efficiency and effectiveness of hydroxyapatite (HA) chromatography. The inventors discovered that desalting a peptide solution before loading the solution on a hydroxyapatite resin significantly increases the HA column capacity to bind peptide. Furthermore, adding an amino acid to the elution buffer significantly increases the peptide recovery from this chromatographic step.

In a further aspect, the invention provides a method for isolating a recombinant peptide having an insect-specific glycosylation pattern from a cell culture. An exemplary method includes removing cellular and other debris from the cell culture to produce a mixture containing the peptide. This mixture is subjected to a “tangential flow filtration (TFF) cascade”, wherein virus, large molecular contaminants and other contaminants are removed, and the mixture is conditioned for downstream purification steps.

The method further includes, adjusting the pH of the conditioned mixture containing the peptide, passing the pH-adjusted mixture over an anion-exchanger (e.g. to further remove viral particles), and collecting one or more eluate fraction containing the peptide. The fraction(s) collected from the anion exchange column are then passed over a cation exchanger and one or more eluate fraction containing the peptide are collected. The collected fraction(s) from the cation exchanger are then subjected to a low-pH hold procedure to affect viral inactivation. The pH of the collected fraction(s) is then raised and the resulting mixture is desalted and subjected to hydroxyapatite (HA) chromatography. One or more eluate fraction containing the peptide is collected. The collected fraction(s) from the hydroxyapatite column are subjected to hydrophobic interaction chromatography (HIC) to further purify the peptide and separate the peptide from a low-molecular weight contaminant. The peptide containing fractions are pooled and optionally filtered to remove viral particles. The resulting product is preferably concentrated and diafiltered into a storage buffer.

In one embodiment, the method further includes glycoPEGylating the extracted peptide and purifying the glycoPEGylated peptide. Glycopegylation methods are art-recognized, see for example, WO 03/031464 to De Frees et al., which is incorporated herein by reference in its entirety.

In one embodiment, the method is used to produce a therapeutic peptide, such as erythropoietin (EPO) and granulocyte colony stimulating factor (GCSF). Alternatively, the method can be used to produce other recombinant peptides such as GNT1, GaIT1, ST3Gal3, CST2, sialidase, GalNAcT2, Core1GalT, ST6GalNAc1, ST3Gal1, and ST3Gal2.

In a further aspect, the invention provides a lipid composition for use in conjunction with a baculovirus expression system. The composition includes an alcohol, a surfactant, a sterol, a detergent, an anti-oxidant, and a lipid source.

Other objects and advantages of the invention will be apparent to those of skill in the art from the detailed description that follows.

### DESCRIPTION OF THE DRAWINGS

FIG. 1A is a RP-HPLC chromatogram of an insect cell culture liquid containing recombinant EPO peptide. The cell culture liquid was supplemented with 1.5% v/v of fresh lipid mixture at the time of infection. The RP-HPLC profile illustrates the quality of the cell culture broth through the noise to (EPO) peak ratio. Batches supplemented with fresh lipids at the time of infection produce a higher quality broth.

FIG. 1B is a RP-HPLC chromatogram of an insect cell culture containing recombinant EPO peptide. The cell culture liquid was not supplemented with lipid at the time of infection. This control culture is characterized by poor baseline resolution and an asymmetric EPO peak, consistent with poor quality broth.

FIG. 1C is a detail of the RP-HPLC chromatogram as shown in FIG. 1A, representing the retention time period between 16 and 20 minutes. This detail shows the EPO peak and surrounding peaks.

FIG. 1D is a detail of the RP-HPLC chromatogram as shown in FIG. 1B, representing the retention time period between 16 and 20 minutes. The detail shows the EPO peak and surrounding peaks.

FIG. 2 is a diagram illustrating an exemplary tangential flow filtration cascade (TFF cascade) employing 100 kDa and 10 kDa molecular weight cut-off membranes.

FIG. 3 is a silver-stained protein gel illustrating the essential removal of a low-molecular weight impurity (labeled “impurity A”) from an EPO containing product mixture by hydrophobic interaction chromatography (HIC) using various HIC resins. The lanes are identified as follows: lane 1: HIC load (UnoSphereS Pool); lane 2: pooled product fractions (Phenyl LS resin); lane 3: flow-through and wash (Phenyl LS resin); lane 5: pooled product fractions (Phenyl 650M resin); lane 6: flow-through and wash (Phenyl 650M resin); lane 8: pooled product fractions (Butyl 4 Sepharose FF resin); lane 10: molecular weight marker; lane 4, lane 7 and lane 9: blank.

FIG. 4A shows the effect of a low-pH hold on EPO peptide recovery in % as determined by RP-HPLC. The figure shows that in this experiment EPO peptide recovery is highest at pH 2.5 (recovery about 80%) and is not related to the sodium chloride concentration in the buffer. The experiment further indicates significant loss of EPO peptide at pH 3 to pH 4.

FIG. 4B shows the effect of a low-pH hold on EPO peptide recovery in % as determined by RP-HPLC. The figure shows a trend of increasing EPO peptide recovery with decreasing pH. In this experiment, the EPO recovery is about 90 % at pH 2.0.

FIG. 5 illustrates EPO peptide breakthrough during hydroxyapatite (HA) chromatography at various HA column loads (mg peptide/mL HA resin). The figure compares the effect of desalted and diluted loads. This graph illustrates that 10% peptide breakthrough is reached before loading 2 mg/mL with a diluted load, while 10 % breakthrough is not reached even with a load of greater than 9 mg/mL when the load is desalted.

FIG. 6 illustrates the effect of glycine addition on the recovery of EPO peptide during hydroxyapatite (HA) chromatography. The figure shows that the recovery of EPO peptide in the main peak is significantly higher when using a buffer containing 20 mM glycine, compared to the recovery when using the same buffer without glycine. The figure also shows that EPO peptide contained in the tail fractions of the EPO peak as well as the EPO peptide retained on the column is reduced.

FIG. 7 is an overall view of an exemplary peptide purification process according to a method of the invention.

### DETAILED DESCRIPTION OF THE INVENTION

Abbreviations

PEG, poly(ethyleneglycol); PPG, poly(propyleneglycol); Ara, arabinosyl; Fru, fructosyl; Fuc, fucosyl; Gal, galactosyl; GalNAc, N-acetylgalactosaminyl; Glc, glucosyl; GlcNAc, N-acetylglucosaminyl; Man, mannosyl; ManAc, mannosaminyl acetate; Xyl, xylosyl; and NeuAc, sialyl (N-acetylneuraminyl); M6P, mannose-6-phosphate; BEVS, baculovirus expression vector system; CV, column volume; NTU, nominal turbidity units; vvm, volume/volume/min.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization are those well known and commonly employed in the art. Standard techniques are used for nucleic acid and peptide synthesis. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well known and commonly employed in the art. Standard techniques, or modifications thereof, are used for chemical syntheses and chemical analyses.

All oligosaccharides described herein are described with the name or abbreviation for the non-reducing saccharide (i.e., Gal), followed by the configuration of the glycosidic bond (α or β), the ring bond (1 or 2), the ring position of the reducing saccharide involved in the bond (2, 3, 4, 6 or 8), and then the name or abbreviation of the reducing saccharide (i.e., GlcNAc). Each saccharide is preferably a pyranose. For a review of standard glycobiology nomenclature see, Essentials of Glycobiology Varki et al. eds. CSHL Press (1999).

Oligosaccharides are considered to have a reducing end and a non-reducing end, whether or not the saccharide at the reducing end is in fact a reducing sugar. In accordance with accepted nomenclature, oligosaccharides are depicted herein with the non-reducing end on the left and the reducing end on the right.

The term “insect cell culture” refers to the in vitro growth and culturing of cell derived from organisms of the Class Insecta. “Insect cell culture” also refers to a cell culture comprising cells of the Class Insecta which have been grown and cultured in vitro.

The term “multiplicity of infection” refers to a measure of the ratio between the number of viral particles and the number of cells to be infected by the viral particles, e.g., number of plaque forming units (pfu) per cell, or viral prticles per cell. The efficiency of infection is influenced by the MOI as well as by the concentration of viral particles and the concentration of cells.

The multiplicity of infection is also a reflection of the average number of viral particles infecting each cell when the cells and viral particles are mixed in order to initiate infection. Indeed, the number of viral particles binding to and infecting any given cell is a random process, therefore there is statistical variation in the number of particles that bind to and infect each cell. The statistical variation follows a normal distribution. Thus, most cells will be infected with a number of virus particles corresponding to the MOI. However, some cells will be infected by more or fewer particles, and some will be infected by no particles at all. The number of cells escaping infection can be calculated using the Poisson distribution. According to the Poisson distribution, the number of cells remaining uninfected at any given MOI is e^{-MOI}.

“Peptide” refers to a polymer in which the monomers are amino acids and are joined together through amide bonds, alternatively referred to as a polypeptide. Additionally, unnatural amino acids, for example, P-alanine, phenylglycine and homoarginine are also included. Amino acids that are not gene-encoded may also be used in the present invention. Furthermore, amino acids that have been modified to include reactive groups, glycosylation sites, polymers, therapeutic moieties, biomolecules and the like may also be used in the invention. All of the amino acids used in the present invention may be either the D- or L-isomer. The L-isomer is generally preferred. In addition, other peptidomimetics are also useful in the present invention. As used herein, “peptide” refers to both glycosylated and unglycosylated peptides. Also included are petides that are incompletely glycosylated by a system that expresses the peptide. For a general review, see, Spatola, A. F., in CHEMISTRY AND BIOCHEMISTRY OF AMINO ACIDS, PEPTIDES AND PROTEfNS, B. Weinstein, eds., Marcel Dekker, New York, p. 267 (1983). The term peptide includes molecules that are commonly referred to as proteins or polypeptides.

A “glycopeptide” as the term is used herein refers to a peptide having at least one carbohydrate moiety covalently linked thereto. It is understood that a glycopeptide may be a “therapeutic glycopeptide”. The term “glycopeptide” is used interchangeably herein with the terms “glycopolypeptide” and “glycoprotein.”

The term “peptide conjugate” refers to species of the invention in which a peptide is conjugated with a modified sugar as set forth herein.

As used herein, the term “modified sugar” refers to a naturally- or non-naturally-occurring carbohydrate that is enzymatically added onto an amino acid or a glycosyl residue of a peptide in a process of the invention. The modified sugar is selected from a number of enzyme substrates including, but not limited to sugar nucleotides (mono-, di-, and tri-phosphates), activated sugars (e.g., glycosyl halides, glycosyl mesylates) and sugars that are neither activated nor nucleotides. The “modified sugar” is covalently functionalized with a “modifying group.” Useful modifying groups include, but are not limited to, PEG moieties, therapeutic moieties, diagnostic moieties, biomolecules and the like. The modifying group is preferably not a naturally occurring, or an unmodified carbohydrate. The locus of functionalization with the modifying group is selected such that it does not prevent the “modified sugar” from being added enzymatically to a peptide.

The term “glycoconjugation” as used herein, refers to the enzymatically mediated conjugation of a modified sugar species to an amino acid or glycosyl residue of a polypeptide, e.g., an erythropoietin peptide prepared by the method of the present invention. A subgenus of “glycoconjugation” is “glyco-PEGylation,” in which the modifying group of the modified sugar is poly(ethylene glycol), an alkyl derivative (e.g., m-PEG) or reactive derivative (e.g., H₂N-PEG, HOOC-PEG) thereof.

The terms “large-scale” and “industrial-scale” are used interchangeably and refer to a reaction cycle or process that produces at least about 250 mg, preferably at least about 500 mg, and more preferably at least about 1 gram of peptide at the completion of a single cycle.

The term, “glycosyl linking group” as used herein refers to a glycosyl residue to which a modifying group (e.g., PEG moiety, therapeutic moiety, biomolecule) is covalently attached; the glycosyl linking group joins the modifying group to the remainder of the conjugate. A “glycosyl linking group” is generally formed by the enzymatic addition of a modified sugar moiety to a glycosyl residue or amino acid of a peptide.

The term “isolated” refers to a material that is substantially or essentially free from components, which are used to produce the material. For peptides and peptide conjugates of the invention, the term “isolated” refers to material that is substantially or essentially free from components which normally accompany the material in the mixture used to prepare the peptide or peptide conjugate. “Isolated” and “pure” are used interchangeably. Typically, isolated peptides or peptide conjugates of the invention have a level of purity expressed as a range. The lower end of the range of purity for the peptide conjugates is about 60%, about 70%, about 75% or about 80% and the upper end of the range of purity is about 70%, about 75% about 80%, about 90% or more.

When the peptide or peptide conjugates are more than about 90% pure, their purities are also preferably expressed as a range. The lower end of the range of purity is about 90%, about 92%, about 94%, about 96% or about 98%. The upper end of the range of purity is about 92%, about 94%, about 96%, about 98% or about 100% purity.

Purity is determined by any art-recognized method of analysis (e.g., band intensity on a silver stained gel, polyacrylamide gel electrophoresis, HPLC, ELISA, or a similar means).

“Essentially each member of the population” as used herein, speaks to the “homogeneity” of the sites on the peptide and to a population of peptide that share a common structure, e.g., a common glycosyl structure.

“Homogeneity” refers to the structural consistency across a population of peptides or across a population of glycosylation site on a peptide. Thus, in a glycopeptide of the invention in which each glycosyl moiety has the same structure the glycopeptide is said to be about 100% homogeneous. Similarly, when a population of glycopeptides of the invention all have glycosyl moieties of the same structure, such that each peptide of the population is essentially of the same molecular species, the population is said to be about 100% homogeneous. Homogeneity is typically expressed as a range. The lower end of the range of homogeneity for the peptide conjugates is about 60%, about 70% or about 80% and the upper end of the range of purity is about 70%, about 80%, about 90% or more than about 90%.

When the peptide conjugates are more than or equal to about 90% homogeneous, their homogeneity is also preferably expressed as a range. The lower end of the range of homogeneity is about 90%, about 92%, about 94%, about 96% or about 98%. The upper end of the range of purity is about 92%, about 94%, about 96%, about 98% or about 100% homogeneity. The homogeneity of the peptide conjugates is typically determined by one or more methods known to those of skill in the art, e.g., gel electrophoresis, liquid chromatography-mass spectrometry (LC-MS), matrix assisted laser desorption mass time of flight spectrometry (MALDITOF), capillary electrophoresis, and the like.

“Substantially uniform glycosylation pattern,” when referring to a glycopeptide species of the invention, refers to the percentage of glycosylation sites on the peptide that have a glycosyl residue of the same structure. For example a peptide that includes multiple glycosylation site may have a glycosyl residue of the same structure present at all of the possible glycosylation sites or even at 90% of the sites or 80% or 75% of the sites. In these instances the peptide would be said to have a “substantially uniform glycosylation pattern”. Alternatively, when a population of glycopeptides share a common glycosylation pattern, the population may be said to have a “substantially uniform glycosylation pattern” when a majority of the peptides in the population represent essentially a single molecular species.

For instance, when glycosylated peptides are isolated from a cell, without further modification, the peptides may include a range of variations in the precise structure of the glycan. However, in an exemplary embodiment, peptides isolated from insect cells according to the method of the invention have a substantially uniform insect glycosylation pattern. This refers to the fact that the majority of peptides, or substantially all of the peptides, in the preparation represent one distinct molecular species. In an exemplary embodiment, a peptide prepared by the method of the invention has a substantially uniform insect glycosylation pattern.

The term “substantially” in the above definitions of “substantially uniform” generally means at least about 40%, at least about 45%, at least about 50%, at least about 55%, at least about 60%, at least about 65%, at least about 70%, 71%, 72%, 73%, 74%, 75%, 76%, 77%, 78%, 79%, at least about 80%, 81%, 82%, 83%, 84%, 85%, 86%, 87%, 88%, 89%, at least about 90%, 91%, 92%, 93%, 94%, 95%, 96%, 97%, 98%, 99%, or even 100% of the acceptor moieties are glycosylated with the expected insect cell specific glycosylation pattern.

The term “insect specific glycosylation pattern” refers to the glycosylation pattern found on mature glycopeptides produced by insect cells. Typically insect cells generate simple N-linked oligosaccharides terminating in mannose (for review, see e.g., Essentials of Glycobiology A. Varki et al. eds, CSHL Press (1999) pgs:32-33). Typically, N-linked glycans produced by insect cell lines produce glycoproteins that at maturity, include a Man₃GIcNAc₂ structure. Fucose units may also be found on the GlcNAc residue that is directly linked to the peptide. A mature peptide emerging from a cell with an “insect specific glycosylation pattern” thus includes one or more glycans having the Man₃GlcNAc₂ structure. Glycopeptides produced in and isolated from insect cell lines according to the methods of the invention have a substantially uniform insect specific glycosylation pattern. This refers to the fact that on substantially all of the peptides all of the glycan structures have the Man₃GlcNAc₂ structure, and are not degraded to e.g., GlcNAc.

The term “loading buffer” refers to the buffer, in which the peptide being purified is applied to a purification device, e.g. a chromatography column or a filter cartridge. Typically, the loading buffer is selected so that separation of the peptide of interest from unwanted impurities can be accomplished. For instance, when purifying the peptide on a hydroxyapatite (HA) column the pH of the loading buffer and the salt concentration in the loading buffer may be selected so that the peptide is initially retained on the column while certain impurities are found in the flow through.

The term “elution buffer”, also called “limit buffer”, refers to the buffer, which is typically used to remove (elute) the peptide from the purification device (e.g. a chromatographic column or filter cartridge) to which it was applied earlier. Typically, the loading buffer is selected so that separation of the peptide of interest from unwanted impurities can be accomplished. Often the concentration of a particular salt (e.g. NaCl) in the elution buffer is varied during the elution procedure (gradient). The gradient may be continuous or stepwise.

The term “controlled room temperature” refers to a temperature of at least about 10° C., at least about 15° C., at least about 20° C. or at least about 25 ° C. Typically, controlled room temperature is between about 20° C. and about 25° C.

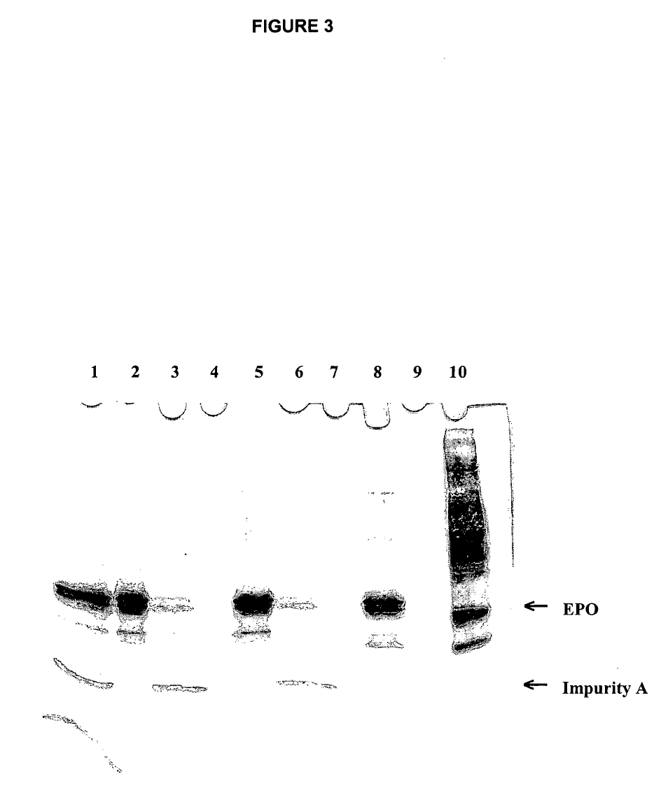

The term “low-molecular weight impurity” refers to a contaminant, which is present in a mixture that also contains a recombinant peptide, wherein the mixture is derived from a cell culture. An exemplary mixture including a low-molecular weight impurity is derived from an insect cell culture. For example, when the peptide EPO is expressed in an insect cell line (e.g. Sf9), the EPO containing mixture isolated from the cell culture contains a low-molecular weight impurity, which is shown in FIG. 3 and is labeled “impurity A”.

Introduction

The present invention provides methods for the large-scale production of peptides and glycopeptides. In one aspect the invention provides a method of generating cell cultures that contain recombinant peptides in improved concentrations and purities. In another aspect, the invention provides novel methods of purifying the recombinant peptide. Combined, these methods form an efficient and cost-effective peptide production process that can provide a high-quality recombinant peptide. In some embodiments, the recombinant peptides so produced are glycopeptides and are further processed to elaborate the structure of their glycosyl residues.

The invention includes a newly discovered infection procedure that provides cell cultures containing a recombinant peptide in high concentration and high purity. The present inventors have discovered that infecting an insect cell culture with a recombinant baculovirus when a lipid mixture is present in the cell culture at the time of infection increases the amount of peptide expressed by the insect cells. In some embodiments, the amount of peptide in the cell culture is increased by about 82% when compared to the amount in a culture not supplemented with the lipid mixture. In other embodiments the amount of recombinant peptide in the cell culture is increased by about 38% when compared to the amount in a culture supplemented with a commercial lipid mixture.

The invention also includes methods of purifying a recombinant peptide. Using a series of ultrafiltration steps, referred to herein as a “tangential flow filtration (TFF) cascade”, the recombinant peptide is removed from the cell culture. This conditioning step delivers the peptide in a concentration and purity that allows subsequent chromatographic purification steps to be more efficient.

Moreover, the invention includes a novel method of inactivating viral particles. In one embodiment the viral inactivation method includes holding the peptide solution at a low pH, at which the peptide of interest is stable. The invention also provides a method of removing a low-molecular weight impurity from the peptide solution. This method employs hydrophobic interaction chromatography and produces a peptide that is unexpectedly pure. In addition, methods of increasing the efficiency and effectiveness of hydroxyapatite (HA) chromatography are provided. The inventors discovered that desalting the HA load containing the peptide before chromatography increases the HA column capacity for bound peptide. Furthermore, adding an amino acid to the elution buffer significantly increases the peptide recovery from this chromatographic step.

The Methods

In a first aspect, the present invention provides an efficient method for the production of peptides and glycopeptides in cell culture.

I. Peptides

The peptide production processes of the present invention can be used to produce any recombinant peptide or glycopeptide. In one embodiment, the peptide or glycopeptide has a molecular weight of about 10 kDa to about 100 kDa. In another embodiment, the peptide or glycopeptide has a molecular weight of about 10 kDa to about 50 kDa, preferably about 10 kDa to about 30 kDa and more preferably about 20 kDa to about 25 kDa.

In one embodiment, the method is used to produce a therapeutic peptide. Exemplary therapeutic peptides include erythropoietin (EPO) and granulocyte colony stimulating factor (GCSF). The method can optionally be used to produce peptides, such as GNT1, GaIT1, ST3Gal3, CST2, Sialidase, GalNAcT2, Core1GalT, ST6GalNAc1, ST3Gal1, and ST3Gal2.

II. Insect Cell Culture

II. a) Cells

The peptides of the current invention can be expressed in any useful cell-line, including bacterial, mammalian and insect cell lines. In an exemplary embodiment, the peptide is expressed in insect cells. Insect cells suitable for use in the present invention are from any order of the class Insecta which can be hosts to recombinant viruses (e.g. baculovirus) or wild-type viruses, and which can grow and produce recombinant peptide products upon infection with the virus in a medium composition of the invention. In an exemplary embodiment, the cells are from the Diptera or Lepidoptera orders.

About 300 insect species have been reported to have nuclear polyhedrosis virus (NPV) diseases, the majority (243) of which were isolated from Lepidoptera (see e.g., Weiss et al., Cell Culture Methods for Large-Scale Propagation of Baculoviruses, In Granados et al. (eds.), The Biology of Baculoviruses: Vol. II Practical Application for Insect Control, pp. 63-87 at p. 64 (1986)). Insect cell lines derived from the following insects are exemplary: Carpocapsa pomonella (preferably cell line CP-128); Trichoplusia ni (preferably cell line TN-368); Autographa californica; Spodoptera frugiperda (preferably cell line Sf9); Lymantria dispar; Mamestra brassicae; Aedes albopictus; Orgyia pseudotsugata; Neodiprion sertifer; Aedes aegypti; Antheraea eucalypti; Gnorimoschema opercullela; Galleria mellonella; Spodoptera littoralis; Drosophila melanogaster, Heliothis zea; Spodoptera exigua; Rachiplusia ou; Plodia interpunctella; Amsacta moorei; Agrotis c-nitrum, Adoxophyes orana, Agrotis segetum, Bombyx mori, Hyponomeuta malinellus, Colias eurytheme, Anticarsia gemmetalis, Apanteles melanoscelus, Arctia caja, and Lymantria dispar.

In an exemplary embodiment, the insect cells are from Spodoptera frugiperda, and in another exemplary embodiment, the cell line is Sf9 (ATCC CRL 1711). Sf9, Sf21, and High-Five insect cells are commonly used for baculovirus expression. Sf9 and Sf21 are ovarian cell lines from Spodoptera frugiperda. High-Five cells are egg cells from Trichoplusia ni. Sf9, Sf21 and High-Five cell lines may be grown at room temperature (e.g. 25 to 27° C.), and do not require CO₂ incubators. Their doubling time is between about 18 and 24 hours.

II. b) Viruses

The insect cell lines cultured to produce the peptides and glycopeptides of the invention are those suitable for the reproduction of numerous insect-pathogenic viruses such as picornaviruses, parvoviruses, entomopox viruses, baculoviruses and rhabdoviruses. In an exemplary embodiment, nucleopolyhedrosis viruses (NPV) and granulosis viruses (GV) from the group of baculoviruses are preferred.

Baculoviruses are characterized by rod-shaped virus particles which are generally occluded in occlusion bodies (also called polyhedra). The family Baculoviridae can be divided in two subfamilies: the Eubaculovirinae comprising two genera of occluded viruses; nuclear polyhedrosis virus (NPV) and granulosis virus (GV), and the subfamily Nudobaculovirinae comprising the nonoccluded viruses.

Methods of preparing and using virus expression systems are generally known in the art. For example, with respect to baculovirus systems, representative references include U.S. Pat. No. 5,194,376, U.S. Pat. No. 5,147,788, U.S. Pat. No. 4,879,236 and Bedard C. et al. (1994) Cytotechnology 15:129-138; Hink W T et al., (1991) Biotechnology Progress 7:9-14; Licari P. et al., (1992) Biotechnology and Bioengineering 39:614-618, each of which is incorporated herein by reference in its entirety.

Thus in one embodiment, the invention includes a baculovirus vector containing a nucleic acid encoding a desired polypeptide. The incorporation of a desired nucleic acid into a baculovirus expression vector may be accomplished using techniques that are well known in the art. For example, such techniques are described in, Sambrook et al. (Third Edition, 2001, Molecular Cloning: A Laboratory Manual, Cold Spring Harbor Laboratory, New York), and in Ausubel et al. (1997), Current Protocols in Molecular Biology, John Wiley & Sons, New York).

II. c) Composition of the Culture Media

Media for culturing insect cells are commercially available. In an exemplary embodiment Sf-900 II, available from Invitrogen, is used to grow insect cell cultures for infection with baculovirus. Sf-900 II medium is optimized to support Sf9 and Sf21 cell growth in both monolayer and suspension applications so that the cells can be used for inter alia Baculovirus Expression Vector System (BEVS) technology.

Protocols for the preparation of insect cell culture media are also known in the art (see e.g., Weiss et al., Cell Culture Methods for Large-Scale Propagation of Baculoviruses, in Granados et al. (eds.), The Biology of Baculoviruses: Vol. II Practical Application for Insect Control, pp. 63-87 at p. 64 (1986)).

In general, insect cell culture media contain inorganic salts e.g., CaCl₂, MgCl₂; sugars e.g., sucrose, maltose; amino acids e.g., L-proline, L-tyrosine; and vitamins e.g., niacin and folic acid. Specific quantities of the various media components are disclosed in Table 1 of Schlaeger, E. (1996) Cytotechnology 20:57-70. This basic media is then supplemented with serum e.g., fetal bovine serum, or alternatively with various lipid compositions.

Lipid Mixture

Lipids are essential for the growth of insect cell cultures in serum free media. The general development of insect cell culture media is reviewed in Schlaeger, E. (1996) Cytotechnology 20: 57-70, which is incorporated herein by reference. Typically, insect cells require a culture medium comprising sterols, fatty acids, amino acids and salts for robust growth.

The present inventors have discovered that, contrary to the teachings of the prior art, the infection of insect cells with recombinant baculovirus encoding a peptide of interest in the presence of a lipid mixture, results in improved yields of the peptide when compared to yields that can be achieved if no lipids are present at the time of infection. Furthermore, in an exemplary embodiment, the quality of the peptide is improved in that the peptides so produced include a substantially uniform glycosylation pattern. The method is particularly useful for the large-scale production of glycopeptides.

In one aspect the present invention provides a lipid mixture that includes an alcohol (e.g. ethanol), a sterol (e.g cholesterol), a surfactant (e.g. block copolymer Pluronic F68), a non-ionic detergent (e.g Tween-80), an antioxidant (e.g. delta-tocopherol acetate), and a lipid source (e.g. cod liver oil).

In one embodiment according to this aspect, the lipid mixture includes an alcohol e.g., ethanol in an amount between about 5% v/v to about 20% v/v, a sterol (e.g cholesterol) in an amount between about 0.02% to about 0.06% w/v, a non-ionic surfactant (e.g. Pluronic F-68) in an amount between about 5% w/v to about 15% w/v, a non-ionic detergent (e.g Tween-80) in an amount between about 0.1% w/v to about 0.3% w/v, an antioxidant (e.g delta-tocopherol acetate) in an amount between about 0.01% w/v to about 0.05% w/v, and a lipid source (e.g cod liver oil) in an amount between about 0.05% w/v to about 0.25% w/v.

In another embodiment the volume of lipid mixture added to supplement the insect cell culture is a volume that is equivalent to between about 0.5% to about 3% v/v. In another embodiment, the volume of lipid mixture added to supplement the insect cell culture is a volume that is equivalent to about 1.0% to about 2.0% v/v, preferably about 1.0% to about 1.5% v/v and, more preferably, about 1.5% v/v.

In another exemplary embodiment, addition of the lipid mixture to the cell culture broth increases the titer of the desired peptide by from about 10% to about 100% compared with the peptide titer when the culture broth is not supplemented with lipid mixture. In another exemplary embodiment, addition of the lipid mixture to the cell culture broth increases the titer of the desired peptide by from about 50% to about 100% and preferably by about 60% to about 100%.

In one embodiment, the lipid mixture is added to the insect cell culture at a time corresponding to between about 0.5 hours to about 3.0 hours prior to infecting. In another embodiment, the lipid mixture is added about 1 hour to about 2 hours and preferably about 1 hour prior to infecting.

In an exemplary embodiment, the lipid mixture is prepared not more than about 48 hours prior to use, and preferably not more than about 24 hours prior to use.

II. d) Viral Infection

Multiplicity of Infection (MO])

The multiplicity of infection, or MOI, represents a measure of the ratio between the number of viral particles and the number of cells to be infected by the viral particles, e.g., number of plaque forming units (pfu) per cell. The efficiency of infection is influenced by the MOI as well as the concentration of viral particles and cells.

The MOI is selected to provide a desired infection efficiency. If the number of viral particles greatly exceeds the number of cells to be infected, the cells are said to be infected at a high MOI. For example, an MOI of 5, wherein there are five times as many viral particles as cells to be infected is considered to be a high MOI. If the number of viral particles is several orders of magnitude less than the number of cells, the MOI is considered to be low.

In one embodiment, the infecting employs a multiplicity of infection between about 10⁻⁸ to about 1.0. In another embodiment, the infecting employs a multiplicity of infection between about 10⁻⁷ to about 0.5. In another embodiment, the infecting employs a multiplicity of infection between about 10⁻⁶ to about 0.2. And, in still another embodiment, the infecting employs a multiplicity of infection of about 0.1 to about 0.2.

Standard multiplicities of infection for baculovirus systems range from between about 0.8 viral particles per cell to about 0.05 particles per cell. However, baculovirus may also be infected at a much lower MOI. Co-pending and co-owned Patent Application No. PCT/US06/01582, filed Jan. 17, 2006, which is incorporated herein by reference in its entirety, discloses that a very low MOI increases yields of recombinant peptide from a baculovirus infection.

In one embodiment, a low MOI is used to initiate infection of insect cells according to the method of the invention. In this embodiment, the MOI is less than or equal to 0.00001 (10⁻⁵) pfu/cell. In another embodiment, the MOI is between 0.00000I(10⁻⁶) to 0.00001(10⁻⁵). In still another embodiment, the MOI is between 0.0000001(10⁻⁷) to 0.000001(10⁻⁶) or between 0.0000001(10⁻⁷) to 0.00001(10⁻⁵). In yet another embodiment, the MOI is between 0.00000001(10⁻⁸) to 0.0000001(10⁻⁷), 0.00000001(10⁻⁸) to 0.000001(10⁻⁶), or 0.00000001(10⁻⁸) to 0.00001(10⁻⁵).

It is well within the ability of the skilled artisan to determine the preferred MOI or the preferred range of MOI best suited for the production of each type or class of polypeptide to be produced according to the method of the invention. Suitable titering methods that can be used to determine the number of viable virus particles in a solution, are known in the art (e.g. standard plaque assay).

II. e) Growth

Insect cell cultures can be grown to high cell densities in bioreactors. Exemplary growth protocols are known in the art, see e.g., Weiss et al. supra.

In an exemplary embodiment, the infected insect cell culture is grown for between about 50 hours to about 100 hours. In another embodiment, the infected insect culture is grown for about 60 to about 70 hours.

III. Isolation of Peptides from Cell Culture

In a second aspect, the current invention provides methods of purifying a recombinant peptide. The protein, which can be expressed in any suitable expression system, is first removed from the cell culture and is then further purified to remove contaminants, such as viral particles and unwanted proteins, using a variety of filtration and chromatographic purification devices.

In baculovirus expression systems, proteins are typically secreted directly from the cell into the surrounding growth media. At the conclusion of a production run, viral particles, whole cells and cellular debris are removed from the culture before the isolation of the peptide from the supernatant begins. These are generally removed by differential centrifugation, continuous centrifugation, by filtration, or by a combination of these methods.

Natural cell death, which occurs during the growth of a culture that produces directly secreted proteins, results in the release of intracellular host cell proteins and produces cellular debris. These contaminants can affect the course of the peptide production run. Indeed, the sub-cellular fragments and host cell proteins released by natural cell death are difficult to remove due to their small size.

Fortunately, insect cell cultures used to prepare recombinant peptides according to exemplary methods of the invention, experience a minimum amount of natural cell death. In an exemplary embodiment, the low level of cell death improves the quality of the culture broth at the end of a production run, which in turn improves the quality of the final peptide product. Furthermore, the improved quality of the culture broth improves the efficiency and cost effectiveness of the production run.

Exemplary steps in a purification cascade of the invention are set forth below. It is to be understood that unless the order of steps is explicitly recited, the exemplary steps are practicable in any desired order.

III. a) Cell Culture Harvest

In order to isolate a peptide of interest from a cell culture, cellular and other debris is removed to produce a suitable feed material for subsequent purification steps. Removing debris can be accomplished using one or more centrifugation steps, one or more filtration steps or a combination of centrifugation and filtration steps.

In an exemplary embodiment, wherein the cell culture volume is small, such as below about 2 liters, batch centrifugation (e.g. bottle centrifugation) can be used. In an exemplary embodiment, the supernatant is further clarified by an appropriate filter or filter train. In another exemplary embodiment, wherein the cell culture volume is from about 10 to about 100 (pilot scale), the debris can be removed directly by a filter train. In another exemplary embodiment, wherein a large-scale production of peptide is desired, cell removal can be accomplished using filtration in addition to centrifugation. In those examples the removal of debris from the cell culture is preferably accomplished using continuous centrifugation followed by filtration.

Centrifugation

The cell culture containing the peptide can be centrifuged using any suitable centrifugation method. In an exemplary embodiment, the peptide purification process of the current invention employs a centrifugation method selected from batch centrifugation, continuous centrifugation and combinations thereof. For large-scale purification processes, centrifuges, which can be operated continuously, are most useful. These allow for the continuous addition of feedstock, the continuous removal of supernatant and the discontinuous, semi-continuous or continuous removal of solids.

In an exemplary embodiment, cell debris is removed by continuous disc-stack centrifugation. Continuous multi-chamber disc-stack centrifuges are known in the art and contain a number of parallel discs providing a large clarifying surface with a small sedimentation distance. In an exemplary embodiment, the sludge is removed through a valve. Disc-stack centrifuges may be operated either semi-continuously or continuously by using a centripetal pressurizing pump within the centrifuge bowl which forces the sludge out through a valve. The capacity and radius of such devices are large and the thickness of liquid is very small, due to the large effective surface area.

In another exemplary embodiment, centrifugation is accomplished using batch centrifugation (e.g. bottle centrifugation).

CaCl₂ is optionally added to the supernatant of the first centrifugation step. The pH of the resulting mixture is then adjusted to about pH 7.5 by adding base (e.g. sodium hydroxide). In an exemplary embodiment, upon addition of base, a precipitate forms. When NaOH is used as the base, the precipitate contains Ca(OH)₂. The precipitate is separated from the liquid (e.g. by filtration or centrifugation). In an exemplary embodiment, this “CaCl₂ precipitation” improves the performance of subsequent ultrafiltration steps.

In another exemplary embodiment, a salt of an organic acid (e.g. citrate) is added to the cell culture (e.g. prior to centrifugation). In an exemplary embodiment, citrate inhibits the activity of degrading enzymes (e.g. endoglycosidases).

III. b) Filtration

Typically, centrifugation effectively removes the bulk of large solids, whole cells, and debris from the cell culture liquid. In addition to this first clarification step, the peptide purification process optionally includes filtration steps, which can be used as a secondary clarification step to remove particulates, virus particles, and to prevent plugging of downstream processing equipment such as membrane filters and ultrafiltration devices.

Depth Filtration

The purification process of the invention optionally includes a depth-filtration step. Depth filtration is effective in removing residual cellular debris and other small particles. Depth filters retain contaminants using two major types of interactions between filters and contaminant particles. Particles are retained due to their size, and may also be retained due to adsorption to the filter material. Molecular and/or electrical forces between the particles and the filter material attract and retain these entities within the filter.

Depth filtration devices are known in the art. In an exemplary embodiment, the filter material is composed of a thick and fibrous cellulose structure with inorganic filter aids such as diatomaceous earth (DE) particles embedded in the openings of the fibers. This construction results in a large internal surface area, which is key to particle capture and filter capacity based on the described retention mechanisms. In another exemplary embodiment a positively charged depth filter is used.

Depth filtration can be accomplished using one or more depth filters. In an exemplary embodiment, two or more depth filters are combined into one multi-layered filter. In one example two filters are used in which the second (downstream) filter is of tighter grade. In an exemplary embodiment a depth filtration step is used subsequent to initial centrifugation of the cell culture liquid.

Membrane Filtration

In another embodiment, the peptide purification process further includes one or more membrane filtration steps to remove small particles. Exemplary membrane filters have a pore size of about 0.1 μm to about 0.5 μm, preferably about 0.1 μm to about 0.3 μm, and more preferably about 0.20 μm to about 0.25 μm.

The membrane filter is optionally part of a multi-layered filter or filter train. For example, the membrane filter is combined with one or more depth filter to form a multi-layered filter device. In an exemplary embodiment the membrane filter forms the most downstream layer of the multi-layered filter device or filter train.

III. c) Tangential Flow Filtration (TFF)

Membrane filtration is a separation technique widely used for clarifying, concentrating, and purifying peptides. Tangential flow filtration, or cross-flow filtration, is a pressure driven separation process that uses membranes to separate components in a liquid solution or suspension based on their size and charge differences. During cross-flow separation, a feed stream is introduced into the membrane element under pressure and passed across the membrane surface in a controlled flow path. A portion of the feed passes through the membrane and is called permeate. The portion of the feed that does not cross the membrane is called retentate.

In one aspect the present invention provides a method of purifying a recombinant peptide, wherein the method includes (a) conditioning a mixture containing the peptide using a tangential flow filtration cascade. According to the method, the conditioning occurs prior to subjecting the mixture to chromatographic purification steps. The method is useful for removing baculovirus and other particles from the peptide solution and then concentrating the semi-purified peptide. The conditioning is accomplished by filtering the peptide solution through a set of ultrafiltration (UF) membranes having a molecular weight cut-off (MWCO) between about 5 kDa and about 200 kDa. The TFF cascade can include any number of high and low MWCO membranes. In one exemplary embodiment, the TFF cascade includes two membrane filters, in which the membranes have a MWCO selected according to the size of the peptide being purified. The two membrane filters can have the same or different MWCO.

In one exemplary embodiment, the peptide being purified has a molecular size that is relatively small compared to the size of certain contaminants. In one embodiment, the current invention provides ultrafiltration and diafiltration strategies that are uniquely tailored to separate small peptides from larger contaminants.

In an exemplary embodiment the TFF cascade includes two membrane filters, in which one membrane filter has a MWCO larger than the purified peptide and another membrane filter has a MWCO smaller than the purified peptide.

An exemplary method contains the following steps to condition a mixture that contains the peptide: (i) ultrafiltering the peptide solution across a first ultrafiltration membrane with a MWCO larger than the purified peptide; (ii) ultrafiltering the permeate from step (i) across a second ultrafiltration membrane with a MWCO smaller than the purified peptide; and (iii) collecting the retentate from step (ii). Preferably, the purified peptide flows through the pores of the first ultrafiltration membrane and is contained in the flow-trough (permeate) of this first ultrafiltration step. Larger proteins such as certain degrading enzymes are thus removed. During the second ultrafiltration step the purified peptide does preferably not cross the membrane and is preferably found in the retentate fraction. This allows the peptide to be concentrated and the buffer system to be altered. The buffer system is altered by replenishing the retentate reservoir with the new buffer. During this “diafiltration” step the original buffer is gradually diluted with the new “diafiltration” buffer.

Ultrafiltration Using a Membrane with a Large MWCO

In an exemplary embodiment, the purification process is initiated by filtering the TFF feed across a first membrane to produce a permeate stream while avoiding the formation of a retentate stream. In an exemplary embodiment, filtration is effected using a transmembrane pressure between about 1 and about 30 psi and a UF filter membrane with a MWCO of between about 75 kDa to about 125kDa and preferably about 100 kDa. The ultrafiltration membrane retains baculovirus particles and other large molecular contaminants, such as larger proteins, while permitting passage of the purified peptide.

In another exemplary embodiment, the membrane utilized in this ultrafiltration step is a member selected from cellulose acetate, regenerated cellulose, and polyethersulfone. Suitable membranes include those, in which the membrane polymer is chemically modified. In a preferred embodiment, the membrane is regenerated cellulose.

Ultrafiltration Using a Membrane with a Small MWCO

In an exemplary TFF cascade, the feed is passed through an ultrafiltration membrane with a MWCO suitable to concentrate the purified peptide. To concentrate a sample, the membrane is chosen to have a MWCO that is substantially lower than the molecular weight of the purified peptide. In general, the ultrafiltration membrane is selected to have a MWCO that is 3 to 6 times lower than the molecular weight of the peptide to be retained by the membrane. If the flow rate or the processing time is of major consideration, selection of a membrane with a MWCO toward the lower end of this range (e.g. 3×) will yield higher flow rates. If recovery of peptide is the primary concern, a tighter membrane (e.g. 6×) is selected (typically with a slower flow rate).

In another exemplary embodiment, filtration is effected using a transmembrane pressure between about 1 and about 30 psi and a filter membrane with a MWCO of between about 5 kDa to about 15 kDa, and preferably 10 kDa. The second filtration step produces a retentate stream and a permeate stream. The retentate is recycled to a reservoir for the peptide solution feed under conditions of essentially constant peptide concentration in the feed by adding a buffer solution to the retentate.

The surface area of the filtration membrane used will generally depend on the amount of peptide to be purified. The membrane may be made of a low-binding material to minimize adsorptive losses and is preferably durable, cleanable, and chemically compatible with the buffers to be used. A number of suitable membranes are commercially available, including, e.g., cellulose acetate, regenerated cellulose and polyethersulfone membranes. Suitable membranes include those in which the membrane polymer is chemically modified. In an exemplary embodiment the membrane is regenerated cellulose.

The flow rate will be adjusted to maintain a constant transmembrane pressure. Generally, filtration will proceed faster with higher pressures and higher flow rates, but higher flow rates may also result in damage to the peptide or loss of peptide due to passage through the membrane. In addition, various TFF devices may have certain pressure limitations on their operation, and the pressure is adjusted according to the manufacturer's specification. In an exemplary embodiment, the pressure is between about 1 to about 30 psi, and in another exemplary embodiment the pressure is between about 8 psi to about 10 psi. Typically, the circulation pump is a peristaltic pump or diaphragm pump in the feed channel and the pressure is controlled by adjusting the retentate valve.

Subsequent to a filtration step or at the conclusion of the TFF cascade, the retentate is collected. Water or an aqueous buffer (e.g. diafiltration buffer) may be used to wash the membrane filter and recover any peptide retained by the membrane. The wash liquid may be combined with the original retentate containing the concentrated peptide. The retentate is optionally dialyzed against a buffer such as TRIS or HEPES before the partially purified peptide is subjected to subsequent purification steps, such as anion exchange chromatography.

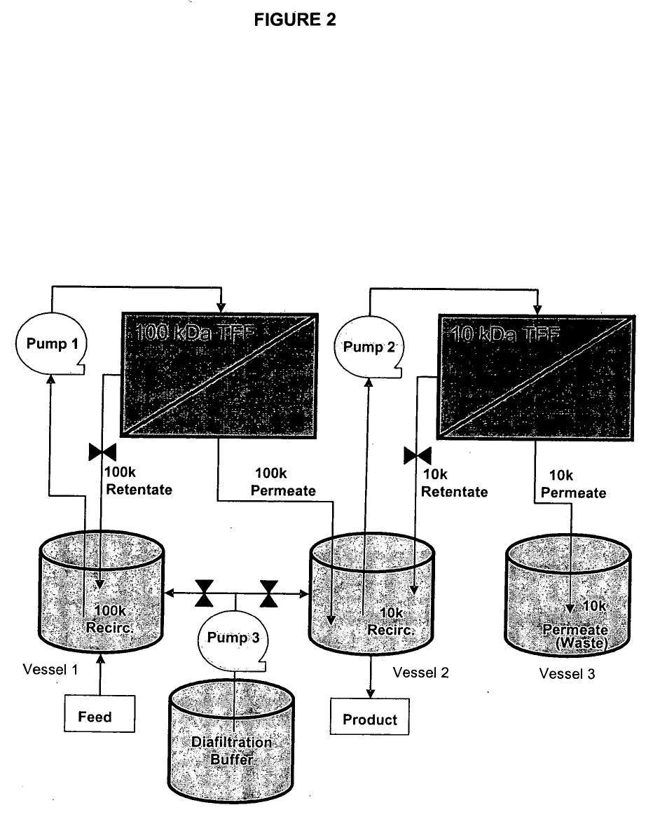

An exemplary TFF cascade is illustrated in FIG. 2. In this example, a feed stream is pumped into the first membrane element (100 kDa TFF) and the 100 kDa permeate is collected in a reservoir (vessel 2). The peptide containing solution is then pumped from vessel 2 into the second membrane element (10 kDa TFF). The 10 kDa permeate from this second filtration step is collected in vessel 3. The retentate may be reintroduced into vessel 2 through a 10 kDa retentate stream. Vessel 4 contains buffer, which is used to refurbish the buffer content in vessel 1 and vessel 2 as needed.

The use of cross-flow filtration (e.g. ultrafiltration and diafiltration) prior to purification of the peptide by chromatographic means, has several unexpected advantages. First, a large part of the viral particles are removed early in the purification process. Second, the overall performance of the peptide purification process is increased. Due to the removal of large-molecular weight contaminants early in the process, the performances of downstream purification steps are significantly increased. Smaller membrane areas and smaller chromatography columns are needed in subsequent purification procedures due to generally cleaner loads.

In addition, removing degrading enzymes from the peptide solution early in the process increases the stability of the peptide during the process and overall yields are thus improved. Due to increased stability of the peptide, subsequent purification steps can optionally be performed at controlled room temperature, eliminating the need to perform the entire purification process in a cold-room facility. Short-term storage of purified peptide (e.g. overnight hold) before shipment and further processing becomes possible.

III. d) Chromatographic Purification of Recombinant Peptides

A variety of recognized chromatographic techniques, such as size exclusion chromatography (gel filtration), ion exchange chromatography, hydrophobic interaction chromatography (HIC), affinity chromatography and mixed-mode chromatography, such as hydroxyapatite chromatography are used for the isolation of peptides and proteins. In an exemplary embodiment, the peptide purification process of the invention employs a combination of several chromatographic techniques. The order in which these steps are performed is dependent on the nature of the peptide being purified and the nature of the contaminants to be removed.

Suitable techniques for the practice of the invention separate the peptide of interest from a variety of contaminants on the basis of charge, degree of hydrophobicity, and/or size. Different chromatographic resins and membranes are available for each of these techniques, allowing accurate tailoring of the purification scheme to the particular peptide being purified.

In one chromatographic technique, the components in a mixture interact differently with the column material and move at different rates along the column length, achieving a physical separation that increases as they pass further down the column. In another chromatographic technique, components of the mixture, including the peptide of interest, adhere selectively to the separation medium, while other components are found in the flow-through. The initially retained components are then eluted differentially by varying the composition of the solvent or buffer system. In another approach, the desired components are found in the flow-through while impurities are retained on the column and thus removed from the mixture.

Ion Exchange Chromatography

Anion and cation exchange chromatography are known in the art. Ion exchange chromatography separates compounds based on their net charge. Ionic molecules are classified as either anions (having a negative charge) or cations (having a positive charge). Some molecules (e.g., proteins) may have both anionic and cationic group. A positively charged support (anion exchanger) will bind a compound with an overall negative charge. Conversely, a negatively charged support (cation exchanger) will bind a compound with an overall positive charge. Ion exchange matrices can be further categorized as either strong or weak exchangers. Strong ion exchange matrices are charged (ionized) across a wide range of pH levels. Weak ion exchange matrices are ionized within a narrow pH range. The ionic groups of exchange columns are covalently bound to the gel matrix and are compensated by small concentrations of counter ions, which are present in the buffer. The most common ion exchange chemistries include: quaternary ammonium residues (Q) for strong anion exchange, diethylaminoethyl residues (DEAE) for weak anion exchange, sulfonic acid (S) for strong cation exchange and carboxymethyl residues (CM) for weak cation exchange.

When adding a sample to the column, an exchange with the weakly bound counter ions takes place. The size of the sample volume in ion exchange chromatography is of secondary importance as long as the initial solvent is of low eluting strength, so as not to allow the sample components to proceed through the column. Under such conditions, the sample components are preferably collected at the top of the column. When the gradient is begun with the addition of a stronger eluting mobile phase, the sample components begin their separation. If poor separation is observed, it might be improved by a change in the gradient slope. If the peptide does not bind to the column under the selected conditions, the composition and/or the pH of the starting buffer should be changed. The buffer system can further be optimized by choosing different buffer salts since each buffer composition solvates the ion exchanger and the sample components uniquely.

In general, any conventional buffer system with a salt concentration of about 5 mM up to about 50 mM can be used for ion exchange chromatography. However, positively charged buffering ions are used for anion exchangers and negatively charged ones are used for cation exchangers. Phosphate buffers are generally used on both exchanger types. Typically, the highest salt concentration that permits binding of the peptide of interest is used as the starting condition. All buffers are prepared from MilliQ-water and filtered (0.45 or 0.22 [m filter).

Anion Exchange Chromatography

In an exemplary embodiment a sample containing the peptide of interest is loaded onto an anion exchanger in a loading buffer comprising a salt concentration below the concentration at which the peptide would elute from the column. The pH of the buffer is selected so that the purified peptide is retained on the anion exchange column. Changing the pH of the buffer alters the charge of the peptide, and lowering the pH value shortens the retention time with anion exchangers. The isoelectric point (pI) of a protein is the pH at which the charge of a protein is zero. Typically, with anion exchangers the pH value of the buffer is kept 1.5 to 2 times higher than the pI value of the peptide of interest. Alternatively, the anion exchange conditions are selected to preferentially bind impurities, while the purified peptide is found in the flow-through.

For weak anion-exchange resins, a low conductivity solution is used, whereas for stronger anion-exchange resins, a high conductivity solution is used. The column is then washed with several column volumes (CV) of buffer to remove those substances that bind weakly to the resin. Fractions are then eluted from the column using, for example, a saline gradient according to conventional methods. The salt in the solution competes with the protein in binding to the column and the protein is released. Components with weak ionic interactions elute at a lower salt concentration than components with a strong ionic interaction. Sample fractions are collected from the column. Fractions containing high levels of the desired peptide and low levels of impurities are pooled or processed separately.

The anion exchangers used in the process of the current invention are employed to separate the purified peptide from contaminants such as viral particles, particulates, proteins/peptides and DNA molecules. An exemplary anion exchanger of the invention is selected from quaternary ammonium resins and DEAE resins. In one embodiment, the anion exchanger is a quaternary ammonium resin (e.g. Mustang Q ion exchange membrane, Pall Corporation).

Cation Exchange Chromatography

In an exemplary embodiment a sample containing the peptide of interest is loaded onto a cation exchange resin in a loading buffer comprising a salt concentration below the concentration at which the peptide would elute from the column.

The pH of the buffer is selected so that the peptide of interest is retained on the cation exchange resin. Changing the pH of the buffer alters the charge of the peptide and increasing the pH of the buffer shortens the retention times with cation exchangers. Typically, cation exchanges are performed at 1.5 to 2 pH units below the peptide's pl. Alternatively, the cation exchange conditions are selected to preferentially bind impurities, while the purified peptide is found in the flow-through.

The column is then washed with several column volumes of buffer to remove those substances that bind weakly to the resin. Fractions are then eluted from the column using a salt gradient according to conventional methods. Sample fractions are collected from the column. One or more fraction containing high levels of the desired peptide and low levels of impurities are collected, and optionally pooled.

In an exemplary embodiment the cation exchangers used in the process of the current invention provide one of the primary purification steps of the purification process. In one embodiment, the cation exchanger removes the majority of undesired proteins from the mixture, which contains the peptide of interest.

In an exemplary embodiment, cation exchange resins of use in the invention are selected from sulfonic acid (S) and carboxymethyl (CM) supports. In one embodiment, the cation exchanger is a sulfonic acid support (e.g. UNOsphereS, Bio-Rad Laboratories).

The ion exchangers used in the methods of the invention are optionally membrane adsorbers rather than chromatographic resins or supports. In an exemplary embodiment, the membrane adsorber is a cation exchanger. In another exemplary embodiment the membrane adsorber is a sulfonic acid (S) cation exchanger (e.g. SartobindS, Sartorius A G). The membrane adsorber is optionally disposable.

Mixed-Mode or Pseudo-Affinity Chromatography

In an exemplary embodiment, the peptide purification process of the invention includes mixed-mode or pseudo-affinity chromatography, such as hydroxyapatite (HA) chromatography. HA chromatography is an effective purification mechanism, providing biomolecule selectivity, complementary to ion exchange or hydrophobic interaction techniques. Hydroxyapatite chromatography is known in the art.

Exemplary hydroxyapatite sorbents are selected from ceramic and crystalline hydroxyapatite materials. Ceramic hydroxyapatite sorbents are available in different particle sizes (e.g. type 1, Bio-Rad Laboratories). In an exemplary embodiment the particle size of the ceramic hydroxyapatite sorbent is between about 20 μm and about 180 μm, preferably about 60 to about 100 μm, and, more preferably about 80 μm.

In one embodiment, the hydroxyapatite sorbent is composed of cross-linked agarose beads with microcrystals of hydroxyapatite entrapped in the agarose mesh. Optionally, the agarose is chemically stabilized (e.g. with epichlorohydrin under strongly alkaline conditions). In one exemplary embodiment, the hydroxyapatite sorbent is HA Ultrogel (Pall Corporation).

The selection of the flow velocity used for loading the sample onto the hydroxyapatite column, as well as the elution flow velocity depends on the type of hydroxyapatite sorbent and on the column geometry. In one exemplary embodiment, at process scale, the loading flow velocity is selected from about 30 to about 900 cm/h, from about 150 to about 900 cm/h, preferably from about 500 to about 900 cm/h and, more preferably, from about 600 to about 900 cm/h.

In an exemplary embodiment, the pH of the elution buffer is selected from about pH 7 to about pH 9, and preferably from about pH 7.5 to about pH 8.0.

In one aspect the present invention provides a method of purifying a recombinant peptide by hydroxyapatite chromatography. The method includes the following steps: (a) desalting a mixture containing the peptide, forming a desalted mixture (e.g. by gel filtration) that has a salt conductivity, which is sufficiently low to increase the peptide-binding capacity of the hydroxyapatit resin; (b) applying the desalted mixture to a hydroxyapatite resin; (c) washing the hydroxyapatit resin, thereby eluting unwanted components from the resin; (d) eluting the peptide from the resin with an elution buffer that optionally contains an amino acid; and (e) collecting one or more eluate fraction containing the peptide.

Desalting

In one embodiment, the mixture containing the peptide of interest is desalted prior to subjecting the mixture to HA chromatography. The desalting step increases the capacity of the HA column to bind the peptide of interest. In one embodiment, the HA column capacity (amount of peptide per liter of HA resin), increases with decreasing salt conductivity of the load, which contains the peptide.

In an exemplary embodiment, in which the load is desalted, the mass loading of peptide per liter of HA resin is from about 1 to about 25 g/L, from about 1 to about 20 g/L, preferably from about 1 to about 15 g/L and more preferably from about 1 to about 10 g/L.

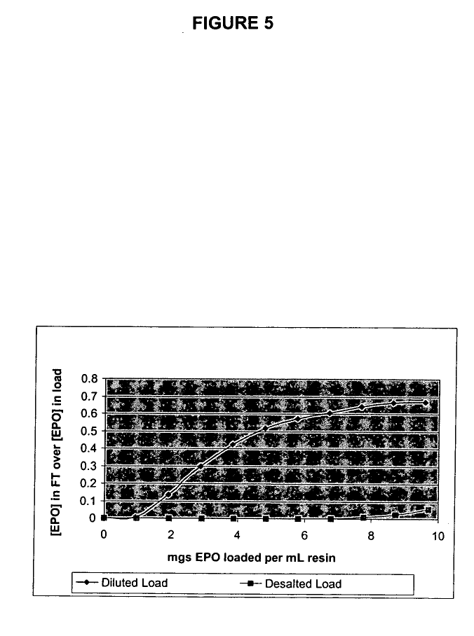

In an exemplary embodiment, in which the peptide being purified is EPO, desalting the loading buffer increases the HA column capacity as shown in FIG. 5. In an exemplary embodiment, the peptide-binding capacity, at which the breakthrough of EPO peptide is less than 10%, is at least about 2 g/L, at least about 4 g/L, at least about 6 g/L, at least about 8 g/L and preferably at least about 10 g/L.

In another exemplary embodiment, the conductivity of the load can be decreased using a method selected from desalting and diluting.

In an exemplary embodiment, the conductivity of the loading buffer is lowered by desalting and preferred conductivities are from about 0.1 to about 4.0 mS/cm, preferably from about 0.1 to about 1.0 mS/cm, more preferably from about 0.1 to about 0.6 mS/cm and, still more preferably, from about 0.1 to about 0.4 mS/cm.

Desalting of peptide solutions is achieved using membrane filters wherein the membrane filter has a MWCO smaller than the peptide/protein of interest. The peptide/protein is found in the retentate and is reconstituted in a buffer of choice. However, when purifying peptides of relatively low molecular weight (e.g. EPO), the MWCO of the membrane used for desalting must be relatively small in order to avoid leaking of the peptide through the membrane pores. However, filtering a large volume of liquid through a small MWCO membrane (e.g. with a pore size of about 5 kDa), typically requires large membrane areas and the filtering process is time consuming.

Therefore, in one embodiment, desalting of the HA chromatography load is accomplished using size-exclusion chromatography (e.g. gel filtration). The technique separates molecules on the basis of size. Typically, high molecular weight components can travel through the column more easily than smaller molecules, since their size prevents them from entering bead pores. Accordingly, low-molecular weight components take longer to pass through the column. Thus, low molecular weight materials, such as unwanted salts, can be separated from the peptide of interest.

In an exemplary embodiment, the column material is selected from dextran, agarose, and polyacrylamide gels, in which the gels are characterized by different particle sizes. In another exemplary embodiment, the material is selected from rigid, aqueous-compatible size exclusion materials. An exemplary gel filtration resin of the invention is Sepharose G-25 resin (GE Healthcare).

In an exemplary embodiment, desalting is performed subsequent to cation exchange chromatography (e.g. after UnoSphere S chromatography).

Addition of an Amino Acid to the Elution Buffer

In one embodiment, an amino acid is added to the elution buffer, which is used to elute the peptide of interest from the HA resin. In an exemplary embodiment the amino acid is added to the elution buffer at a final concentration of about 5 mM to about 50 mM, about 10 mM to about 40 mM, preferably about 15 mM to about 30 mM and, more preferably, about 20 mM.

In one embodiment, the addition of an amino acid (e.g. glycine) to the elution buffer increases the step recovery of peptide from HA chromatography when compared to the recovery obtained without the addition of an amino acid. In an exemplary embodiment, the recovery of peptide is increased by addition of the amino acid at least about 1% to about 20%, by at least about 1% to about 15%, by at least about 1% to about 10%, preferably by at least about 1% to about 7% and, more preferably, by about 5%.

In another exemplary embodiment, the addition of an amino acid (e.g. glycine) causes the elution peak of the purified peptide to be sharper. Thus, less peptide is recovered in the tail fractions of the peak and more peptide is recovered in the main peak. In another exemplary embodiment, the addition of an amino acid (e.g. glycine) does not decrease the purity of the product from HA chromatography.

In an exemplary embodiment, the amino acid is glycine. In a preferred embodiment, glycine is added to the elution buffer at a final concentration of 20 mM.

Hydrophobic Interaction Chromatograihy (HIC)

Hydrophobic interaction chromatography (HIC) is a liquid chromatography technique that separates biomolecules based on differences in their surface hydrophobicity. Hydrophobic amino acids exposed on the surface of a polypeptide, can interact with hydrophobic moieties on the HIC matrix. The amount of exposed hydrophobic amino acids differs between polypeptides and so does the ability of polypeptides to interact with HIC gels. Hydrophobic interaction between a biomolecule and the HIC matrix is enhanced by high ionic strength buffers, and HIC of biomolecules is typically performed at high salt concentrations. The elution of the peptide of interest from the column is then initiated by decreasing salt gradients.

HIC media are distinguished by the hydrophobic moiety that they carry, by the particle size (e.g. bead size), and the density of the hydrophobic moieties on the HIC matrix (e.g. low substitution or high substitution). In an exemplary embodiment, the hydrophobic moieties of the column matrix are members selected from alkyl groups, aromatic groups and ethers. Exemplary hydrophobic alkyl groups include lower alkyl groups, such as n-propyl, isopropyl, n-butyl, and n-octyl. Exemplary aromatic groups include substituted and unsubstituted phenyl.

In another exemplary embodiment the matrix of the HIC medium is a member selected from agarose, sepharose (GE Healthcare), polystyrene, divinylbenzene, and combinations thereof. Exemplary HIC resins include Butyl Fast Flow and Phenyl Fast Flow (both GE Healthcare) in either low or high substituted versions. In a preferred embodiment, the HIC resin is Butyl Sepharose Fast Flow (GE Healthcare).

In another exemplary embodiment, the buffer in which the product is applied to the HIC column contains salts, such as sodium acetate (NaOAc), sodium chloride (NaCl), and sodium sulfate (Na₂SO₄). The concentration ranges for these and other salts are generally optimized for each type of HIC resin to affect optimal binding of the peptide.

In an exemplary embodiment, the concentration of sodium sulfate in the loading buffer is about 100 mM to about 1M, preferably about 300 mM to about 800 mM and, more preferably, about 400 mM to about 600 mM. In another exemplary embodiment, the concentration of NaCl in the buffer is about 100 mM to about 1M, preferably about 200 mM to about 400 mM and, more preferably, about 200 mM to about 300 mM. In yet another exemplary embodiment the concentration of NaOAc in the loading buffer is about 1 mM to about 50 mM, preferably about 5 mM to about 20 mM and, more preferably, about 5 mM to about 15 mM.

In another exemplary embodiment, the buffer in which the product is applied to the HIC column has a pH of about 4.0 to about 6.0, preferably about 4.5 to about 5.5 and, more preferably, about 5.0.

In yet another exemplary embodiment, the product is eluted from the HIC resin with a sodium acetate buffer at a pH of about 5.0 to about 7.5. Exemplary elution buffer systems include TRIS buffer and HEPES buffer. Optionally, the elution buffer does not contain sodium sulfate. In a further exemplary embodiment the elution buffer contains ethanol in an amount of about 5% to about 10% v/v.

In one aspect, the present invention provides a method of separating a peptide from an impurity, wherein the impurity has a molecular weight smaller than the peptide by hydrophobic interaction chromatography. The method comprises: (a) applying a mixture containing the peptide and the impurity to a suitable hydrophobic interaction chromatography resin; (b) eluting the impurity from the resin; (c) eluting the peptide from the resin; and collecting one or more eluate fraction containing the peptide.

In one preferred embodiment, HIC is employed as an orthogonal method of purification to remove impurities that are difficult to remove using other means, and preferably those that have a smaller molecular weight than the peptide being purified.

In an exemplary embodiment, the content of the low-molecular weight impurity (e.g. impurity A in FIG. 3) is reduced by at least 50% of its content before HIC. In another exemplary embodiment, the impurity is reduced by at least 60%, preferably at least 80% and, more preferably, at least 90% of its original content. In certain preferred embodiments the content of the impurity in the mixture processed by HIC is reduced by at least 91%, at least 92%, at least 93%, at least 94%, at least 95%, at least 96%, at least 97%, at least 98% or at least 99%.

In an exemplary embodiment, HIC purification of partially purified EPO yields a product which is essentially free of a low-molecular weight impurity (impurity A) as illustrated in FIG. 3. Although the molecular weight of recombinant EPO is essentially similar to the molecular weight of impurity A, this purification step is particularly effective in separating EPO from impurity A. During HIC chromatography impurity A is found in the flow through, while EPO is initially retained on the HIC column.

In an exemplary embodiment, HIC is performed subsequent to hydroxyapatite (HA) chromatography. Performing the two chromatographic steps in this order increases the recovery of peptide after HIC and requires limited conditioning of the buffer system prior to HIC. In an exemplary embodiment, the pH of the hydoxyapatite product pool is lowered to about 5.0 to about 5.5 by addition of an organic acid (e.g. acetic acid). Sodium sulfate is then added to a concentration of about 500 mM to about 1.0 M, preferably about 500 mM in order to condition the partially purified peptide for hydrophobic interaction chromatography.

III. e) Viral Inactivation

The peptide purification process of the current invention includes one or more viral inactivation steps in order to inactivate enveloped and non-enveloped virus particles that may be present in the mixture. This is particularly important when the final product is intended for use in living organisms. Pathogenic viruses are removed to render the product safe for use in humans. Removal of virus particles may be accomplished using a combination filtration and chromatographic steps. Inactivation of enveloped viruses may be accomplished chemically, e.g. by addition of a detergent. Inactivation of remaining viruses may be accomplished through a low pH hold procedure.

Viral Inactivation Using a Detergent

In one exemplary embodiment viral inactivation involves the addition of a detergent to the partially purified peptide solution. In an exemplary embodiment, the detergent is TritonX (e.g. TritonX-100). In a further exemplary embodiment, TritonX-100 is added to inactivate enveloped viruses.

In another exemplary embodiment, the detergent is added at a final concentration of about 0.01% to about 0.1% v/v, preferably about 0.04% to about 0.06% v/v, and, more preferably at a final concentration of about 0.05% v/v. In one exemplary embodiment the detergent is added to the partially purified peptide solution after purification by anion exchange chromatography (e.g. Mustang Q).

Viral Inactivation by a Low-pH Hold Procedure

It is known in the art that many viruses do not survive a prolonged treatment with a low pH medium. However, when purifying peptides and proteins, the pH of the buffer system is generally crucial in maintaining the stability of the product. Many proteins and peptides cannot withstand a pH well below 7.0.

In one aspect, the present invention provides a method of inactivating viruses in a mixture containing the peptide of interest. The method comprises: (a) lowering the pH of the mixture containing the peptide to a pH below pH 7; (b) maintaining the low pH of step (a) for a selected period of time (e.g. about 1 hour); and raising the pH of the mixture containing the peptide to a pH suitable for further processing.

In an exemplary embodiment, the pH of step (a) is lowered to about pH 2 to about pH 4, preferably to about pH 2 to about pH 3 and, more preferably, to about pH 2 to about pH 2.5. In one preferred embodiment, the pH of the product solution is lowered to between about pH 2.2 to about pH 2.5.

In a further exemplary embodiment, the pH of the peptide solution is maintained at the low pH (e.g. about pH 2.2) for at least about 30 min to at least about 2 hours, preferably at least about 1 hour, before the pH is raised.

In another exemplary embodiment, the pH of the product solution is lowered while the peptide solution has controlled room temperature.

In one exemplary embodiment, the pH of the peptide solution is adjusted using acids, which are suitable for biological applications. Exemplary acids include organic acids, inorganic acids and combinations thereof. In an exemplary embodiment the organic acid is a member selected from acetic acid, citric acid, lactic acid, oxalic acid and succinic acid. In another exemplary embodiment the inorganic acid is a member selected from hydrochloric acid (HCl) and phosphoric acid (H₃PO₄).

III. f) Inactivation of Proteases and Glycosidases

In one embodiment, a protease inhibitor, e.g., methylsulfonylfluoride (PMSF), or sodium citrate is added to the partially purified peptide solution to inhibit proteolysis. In another embodiment, a glycosidase inhibitor may be added. This step protects the peptide of interest from degradation. This is particularly useful if the partially purified peptide solution is stored prior to further processing. Antibiotics are optionally added to prevent the growth of adventitious contaminants.

III. g) Viral Clearance and Storage

In an exemplary embodiment, the peptide purification process of the current invention includes an additional ultrafiltration step to affect viral clearance. Typically, this step occurs towards the end of the purification process and employs a membrane with a MWCO larger than the peptide of interest to allow the peptide to flow through the membrane. In an exemplary embodiment, this viral clearance step is introduced into the process after purification of the product by chromatographic means. A number of ultrafiltration membranes are available that are recommended for viral removal. In an exemplary embodiment the membrane is NFP membrane (Millipore Corporation).

In another exemplary embodiment, the peptide purification process of the present invention includes a diafiltration step towards the end of the process. In an exemplary embodiment the diafiltration step is employed to concentrate the sample. In another exemplary embodiment the diafiltration step is employed to alter the buffer. In yet another exemplary embodiment, the new buffer is suitable for storage of the product. In another exemplary embodiment, the diafiltration membrane has a MWCO of about 4kDa to about 10 kDa, preferably about 4 kDa to about 6 kDa and, more preferably about 5 kDa.

The purified product is stored at a low temperature. In an exemplary embodiment the product is stored at about −20° C. at a peptide concentration of about 1 mg to about 2 mg of peptide per mL storage buffer.

III. h) Exemplary Purification Process

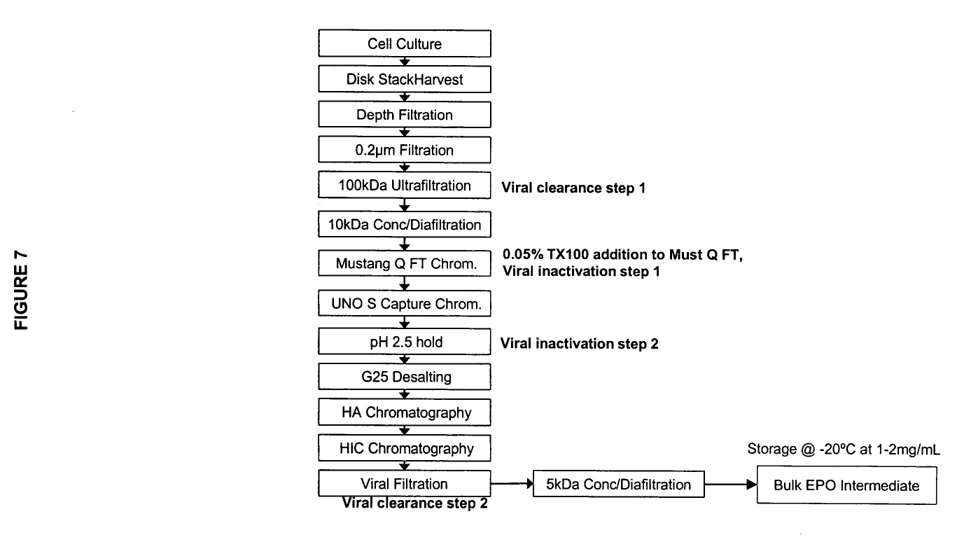

In one aspect of the invention, the peptide of interest is purified from a cell culture using a purification process outlined in FIG. 7. In a first step, cells and cell debris are removed from the cell culture by continuous disk stack centrifugation. The supernatant from this centrifugation step is then filtered through a depth filter and 0.2 μm membrane filter train to further reduce the turbidity of the solution. The resulting material is then subjected to a tangential flow filtration (TFF) cascade. In this step the mixture is first filtered across a 100 kDa TFF membrane. The flow through (permeate) from this first ultrafiltration step is then filtered across a second ultrafiltration membrane with a molecular weight cut-off of 10 kDa. The retentate from this second ultrafiltration step is collected. In an exemplary embodiment, the TFF cascade is used to condition the mixture containing the peptide for subsequent purification steps by removing contaminants with a larger molecular size, and by concentrating the mixture and affecting a buffer exchange.

The resulting mixture containing the peptide is then loaded onto an anion exchanger, such as a Mustang Q anion exchange membrane filter, and the peptide is collected in the flow trough. A non-ionic detergent, such as Triton X-100 is then added to the product pool to a final concentration of about 0.05% (v/v). The mixture is then subjected to cation exchange chromatography (employing e.g UnoSphere S cation exchange resin) and the peptide containing fractions are collected and pooled.

Subsequent to cation exchange the mixture is subjected to a low pH hold procedure to effect viral inactivation. The mixture is then desalted using a size exclusion column (e.g. G25) to lower the salt conductivity of the peptide solution in preparation for hydroxyapatite (HA) chromatography. The desalted mixture is then loaded onto a HA column. The elution pool from the HA column is then conditioned for and subjected to hydrophobic interaction chromatography (HIC). The eluate pool from the HIC column is optionally filtered through a suitable membrane (such as a NFP membrane) for additional viral clearance. The product is diafiltered across a 5kDa TFF membrane and the retentate is reconstituted in a storage buffer to reach a desired peptide concentration (e.g. 1-2 mg/mL).

In an exemplary embodiment according to this aspect, the peptide is produced by expression in an insect cell culture using a baculovirus expression vector system.

In another exemplary embodiment, the recombinant peptide being purified by the above described process is EPO.

IV. Glycopegylation

The glycosylation pattern of the peptides can be elaborated, trimmed back or otherwise modified by methods utilizing enzymes. The methods of remodeling peptides and lipids using enzymes that transfer a sugar donor to an acceptor are discussed in detail in WO 03/031464 to De Frees et al. (published Apr. 17, 2003); U.S. patent application 20040137557 (filed Nov. 5, 2002); U.S. patent application 20050143292 (filed Nov. 24, 2004) and WO 05/051327 (filed Nov. 24, 2004), each of which is incorporated herein by reference in its entirety.

The following examples are provided to illustrate the conjugates, and the methods of the present invention, but not to limit the claimed invention.

### EXAMPLES

### Example 1

### Preparation of a Lipid Mixture for BEVS Expression

1.1 Preparation of Pluronic F-68 solution

A solution of Pluronic F-68 was prepared as follows: 800 mL of deionized H₂O was stirred rapidly. 90 grams of Pluronic F68 were added to the stirred solution and the volume was adjusted to 900 ml with deionized H₂O. In a covered container Pluronic F-68 was allowed to completely solubilize. After solublizing the Pluronic F-68, the solution was transferred to a 37° C. waterbath during preparation of the lipid mixture.

1.2 Preparation of a 100× Lipid Mixture

100 mL of absolute ethanol was warmed to 37° C. while stirring in a covered container. Cholesterol was added to the ethanol, and solubilized. Tween 80 was added to the lipid solution next after the cholesterol, and acts to improve cholesterol solubility. The remaining components of the lipid mixture were then added. The components were added to the ethanol in the amounts indicated below in Table 2. Using a stir plate with a heating element the mixture was heated to 45° C. while stirring in order to solubilize the components resulting in a clear solution.

TABLE 2
Lipid Mixture Components for 100X Stock Solution

| COMPONENT | AMOUNT/1 L | |
|---|---|---|
| Ethanol | 100.00 | mL |
| Cholesterol | 450.00 | mg |
| Tween 80 | 2500.00 | mg |
| Cod Liver Oil | 1700.00 | mg |
| d-Tocopherol Acetate | 300.00 | mg |
| F-68 (10%) | 900.00 | mL |

After preparing the lipid mixture, the F-68 solution was removed from the waterbath and rapidly stirred with a magnetic stir bar. The 100 mL of EtOH/lipid mixture was added dropwise to the rapidly stirring F-68. After addition was complete, the solution was rapidly mixed for another 10-20 minutes while sealed/covered.

The 10% F-68/lipid mixture was then allowed to settle. After disappearance of the bubbles, created by stirring, the mixture was visually inspected for clarity. A clear (slightly opaque) solution was obtained.

The lipid mixture was then sterile filtered using a 0.2 μm non-binding filter. As an example, 10 mL or 15 mL of this 100× lipid mixture are used to supplement 1 liter of insect cell culture medium.

### Example 2

### Effects of Lipid Mixture Addition on EPO Production in Sf9 Insect Cell Cultures

The effect of lipid supplementation on the production of erythropoetin (EPO) was investigated. A commercially available, chemically defined lipid concentrate was compared to the fresh lipid mixture prepared as discribed in Example 1. The fresh lipid mixture was added to the cell culture at 0%, 1.0% and 1.5% v/v. The data shows that the fresh lipid mixture added at the time of infection produced EPO titers in Sf9 cell cultures that were 38% higher than those from cultures supplemented with the commercial lipid mixture. The study also demonstrated that 1.5% lipid supplementation yields an EPO titer that is 82% higher than the control (no lipid addition) and 35% higher than the 1.0% supplementation. Both lipid preparations supplemented at 1.5% produced a cleaner cell culture broth and higher quality EPO. It was also observed that when either lipid mix was added, the drop in cell viability through infection was less than the control (no lipid).

The cell cultures were generated using Sf-900II media from Invitrogen. Multiple components in the lipid mixtures, including cholesterol, Pluronic F-68, and cod liver oil, are already existent in the Sf-900II media. This study looked at the effect on EPO titers and/or quality by supplementing the SF-900II media with additional lipid mixture at the time of infection.

The cell culture batches were performed at the 10-15 L scale in a Biostat C fermenter using the freshly prepared lipid mixture of Example 1, and a commercially available, chemically defined lipid mix. The ingredients of both lipid mixtures are compared in Table 3.

2.1. Materials

SF9 cells originating from working cell banks
Invitrogen Sf-900II media
Commercially available Chemically Defined Lipids
B. Braun Biostat C 20 L Fermenters, Z-1100 H,I,J,E,F
Invitrogen Yeastolate, Ultrafiltrate
Baculovirus
Biosafety Hood,
WAVE Tubing Fuser
WAVE Tubing Sealer
Guava PCA Cell Counter, AE-1038

Getinge Novus I Autoclave, AU-1000

TABLE 3
Comparison of Lipid Mixture Components (100X Stock Solutions)

| Commercial Lipid Mixture | Fresh Lipid Mixture |
|---|---|
| Pluronic F-68 ® (100 g/L) | Pluronic F-68 ® (90 g/L) |
| Tween 80 ® (2.2 g/L) | Tween 80 ® (2.5 g/L) |
| Cholesterol (220 mg/L) | Cholesterol (450 mg/L) |
| DL-α-Tocopherol-Acetate | DL-α-Tocopherol-Acetate (300 mg/L) |
| (70 mg/L) | |
| Linoleic Acid (10 mg/L) | Cod Liver Oil (1.7 g/L) |
| Linolenic Acid (10 mg/L) | Ethanol (100 mL/L) |
| Myristic Acid (10 mg/L) | N/A |
| Oleic Acid (10 mg/L) | N/A |
| Palmitoleic Acid (10 mg/L) | N/A |
| Palmitic Acid (10 mg/L) | N/A |
| Stearic Acid (10 mg/L) | N/A |
| Arachidonic Acid (2 mg/L) | N/A |

2.2 Standard Plaque Assay

The baculovirus particles were titered according to a standard plaque assay. Two 6-well tissue culture plates were placed in a biological safety cabinet for each sample to be assayed, as are two plates for each of the controls. Sf9 cells were diluted to 5×10⁵ cells/mL in a sterile container using pre-warmed Sf-900 II SFM (1×) and mixed gently. The 6-well plates were labeled as follows: Two wells were labeled as “negative control” and two wells were labeled as “positive control,” ensuring two empty wells between samples. Duplicate control plates were similarly labeled. For each sample plate, two wells were labeled for every dilution assayed. Duplicate sample plates were labeled similarly.

Sample dilutions were based on the expected baculovirus titer of the sample. For example, a sample with an expected titer of approximately 1×10⁷ pfu/mL would be assayed at 10⁻⁶, 10⁻⁷ and 10⁻⁸ dilutions.

Two mL of diluted Sf9 cells were added to each labeled well and allowed to attach for a minimum of 1 hour at room temperature. Cells were added along the wall of the well, and were gently pipetted to assure cell suspension. Three mL each of Sf-900 II SFM (1×) were aliquoted into two sterile tubes. Three μl of a positive control viral stock was added to each of the tubes and mixed, to represent a 10⁻³ dilution. For each sample, the appropriate amount of Sf-900 II SFM (1×) was aliquoted into sterile tubes, based on the dilutions that were to be assayed. Samples were prepared in duplicate, and diluted 10-fold so that there was a minimum of 3 mL of each dilution available for assay.

After one hour incubation of the Sf9 cells, except for the negative control wells, media was removed from the wells and 1 mL each of the viral dilution corresponding to the labeled wells was inoculated. Media was added slowly along the wall of the well to minimize dislodging the cell monolayer, and the resultant mixtures were incubated for at least one hour at room temperature. At the end of one hour, an agar/media solution was prepared by diluting 4% Agar 1:4 with Sf-900 Medium (1.3×).

The inoculum was removed from each well of the 6-well plate and 2 mL of the agar/media solution was added rapidly by letting the solution run down the side of each well to which it is added. The plates were then incubated for 45 minutes at room temperature. At the end of the 45 minute incubation, paper towels moistened with approximately 2-4 mL of 5 mM EDTA were wrapped around the plates. The plates were inverted, placed in a sterile bag and incubated at 27° C. for 7-10 days, or until plaques were visible in the positive control wells to the naked eye. After 7-10 days, the plates were unwrapped and 5-7 drops of MTT staining solution were added to each well. Purple color was allowed to develop at room temperature for approximately six hours, or until the entire well is stained purple. Plaques appeared clear against a purple background. The plaques were counted and the numbers recorded. An average is recorded for the duplicate wells at each dilution. The number of plaques between 10-fold dilutions differed by less than a factor of ten. The highest dilution with at least 5 plaques provided a representative number for the plaques formed at that particular dilution.

The viral titer was determined as follows. For example, if the well representing the 10⁻⁸ dilution had 9 colonies, the titer of the viral stock solution is 9×10⁸. The units used are Plaque Forming Units/mL (PFU/mL). Therefore, for example, the viral titer is represented as 9×10⁸ PFU/mL. If there were no plaques present in the negative control and plaques were present in the positive control, then the assay was determined to be valid.

2.3 Study I: Fresh Lipid Addition vs Control (no lipid supplement)

Two experiments were performed for this study. In the first experiment, Sf9 cells were used while the second experiment used GMP Sf9 cells. Also, the MOI used in Experiment I was 0.8, while the second experiment used MOI's of 0.1-0.2. The protocols for these experiments are outlined below, and the results are summarized in Table 8.

Experiment 1

5 liters Sf9 cells with viable cell density (VCD) of 1.8×10⁷ cells/mL and viability of >90% were transferred to fermenter Z-1100J. 7 liters Sf-900II media was added immediately after cell addition. 150 mL fresh lipid mix was prepared according to the protocol in Example 1. The lipid mix was added aseptically to the 12 L cells via the 19 mm head port/septum to give a final lipid mix addition of 1%. Cells were allowed to acclimate with the lipid mix for 1 hour. The cells were infected with 3 L baculovirus at a titer=2.28×10⁷ pfu/mL as determined by the standard plaque assay (see above). Cell infection VCD (viable cell density) was 6×10⁶ cells/mL and >90% viability. The run parameters are outlined in the Table 4 below.

TABLE 4
Summary of Parameters for Experiment 1

| Pre-Infection Parameters | |
|---|---|
| Agitation (rpm)/Impellor Type | 40/pitched blade (45°) |
| Aeration (lpm) | 0.4 |
| Temperature (° C.) | 27.5 |
| DO Setpoint (%) | 60 |
| Post-Infection Parameters | |
| Agitation (rpm) | 60 |
| Aeration (lpm) | 0.6 |
| Temperature (° C.) | 27.5 |
| DO Setpoint (%) | 60 |

Experiment 2A

2 liters (L) Sf9 cells with VCD of 4.7×10⁶ cells/mL and viability of >90% were transferred to fermenter Z-1100H containing 5 liters Sf-900II media. After 4 days, when VCD=6.29×10⁶ cells/mL and viability=78.5%, 100 mL yeastolate was added to increase VCD. The following day, when VCD=8.04×10⁶ cells/mL and viability=74.3%, 225 mL fresh lipid mix (prepared according to the protocol in Example 1) was added aseptically through the 19mm head port/septum on Z-1100H septum to give a final lipid mix addition of 1.5%. Cells were allowed to acclimate to the lipid mix for 1 h. After 1 h, the cells were infected with 200 mL concentrated virus (titer=5.22×10⁷ pfu/mL as determined by the standard plaque assay). An additional 7 L Sf-900II media was added after infection. The run parameters are outlined in the Table 5 below.

TABLE 5
Summary of Parameters for Experiment 2A

| Pre-Infection Parameters | |
|---|---|
| Agitation (rpm)/Impellor Type | 80/marine |
| Aeration (lpm) | 0.3 |
| Temperature (° C.) | 27.5 |
| DO Setpoint (%) | 60 |
| Post-Infection Parameters | |
| Agitation (rpm) | 100-120 |
| Aeration (lpm) | 0.4-0.8 |
| Temperature (° C.) | 27.5 |
| DO Setpoint (%) | 60 |

Experiment 2B

2 liters of Sf9 cells with VCD of 4.1×10⁶ cells/mL and viability of >90% were transferred to fermenter Z-1100J containing 5 L Sf-900II media. After 4 days, when VCD=6.85×10⁶ cells/mL and viability=90.4%, 100 mL yeastolate was added to increases VCD. The following day, when VCD=10.9×10⁶ cells/mL and viability=90.7%, 150 mL fresh lipid mix (prepared according to the protocol in Example 1) was added aseptically through the 19 mm head port/septum on Z-1100J septum to give a final lipid mix addition of 1%. Cells were allowed to acclimate to the lipid mix for 1 hour. After 1 h, the cells were infected with 200 mL concentrated virus (titer=5.22×10⁷ pfu/mL as determined by the standard plaque assay). An additional 7 L Sf-900II media was added after infection. The run parameters are outlined in the Table 6 below.

TABLE 6
Summary of Parameters for Experiment 2B

| Pre-Infection Parameters | |
|---|---|
| Agitation (rpm)/Impellor Type | 40/pitched blade (45°) |
| Aeration (lpm) | 0.3 |
| Temperature (° C.) | 27.5 |
| DO Setpoint (%) | 60 |
| Post-Infection Parameters | |
| Agitation (rpm) | 50-60 |
| Aeration (lpm) | 0.4-0.6 |
| Temperature (° C.) | 27.5 |
| DO Setpoint (%) | 60 |

Experiment 2C:

2 liters of Sf9 cells with VCD of 4.1×10⁶ cells/mL and viability of >90% were transferred to fermenter Z-1100I containing 5 L Sf-900II media. After 4 days, when VCD=8.39×10⁶ cells/mL and viability=82.5%, 100 mL yeastolate was added to increase VCD. The following day, when VCD=7.08×10⁶ cells/mL and viability=81.4%, the cells were infected with 200 mL concentrated virus (titer=5.22×10⁷ pfu/mL as determined by the standard plaque assay). The final lipid mix addition was 0%. An additional 7 L Sf-900II media was added after infection. The run parameters are shown in the Table 7 below.

TABLE 7
Summary of Parameters for Experiment 2C

| Pre-Infection Parameters | |
|---|---|
| Agitation (rpm)/Impellor Type | 70/marine |
| Aeration (lpm) | 0.3 |
| Temperature (° C.) | 27.5 |
| DO Setpoint (%) | 60 |
| Post-Infection Parameters | |
| Agitation (rpm) | 70-120 |
| Aeration (lpm) | 0.4-0.8 |
| Temperature (° C.) | 27.5 |
| DO Setpoint (%) | 60 |

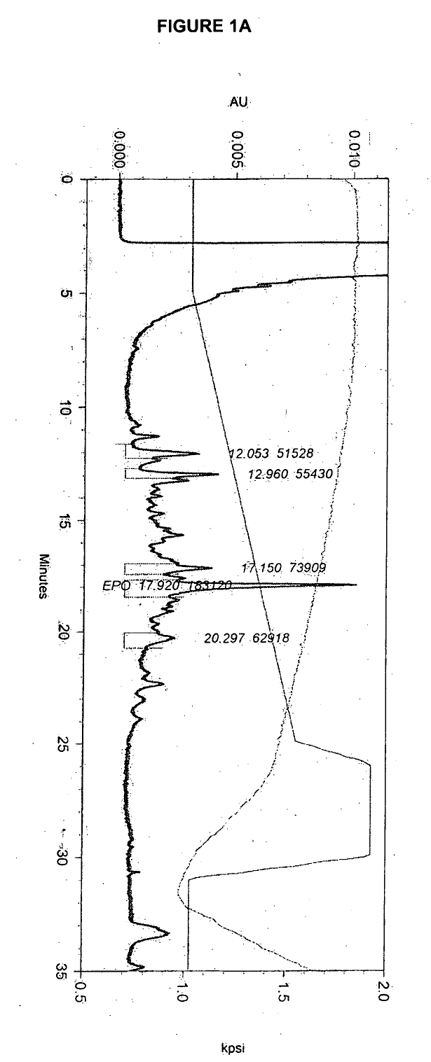

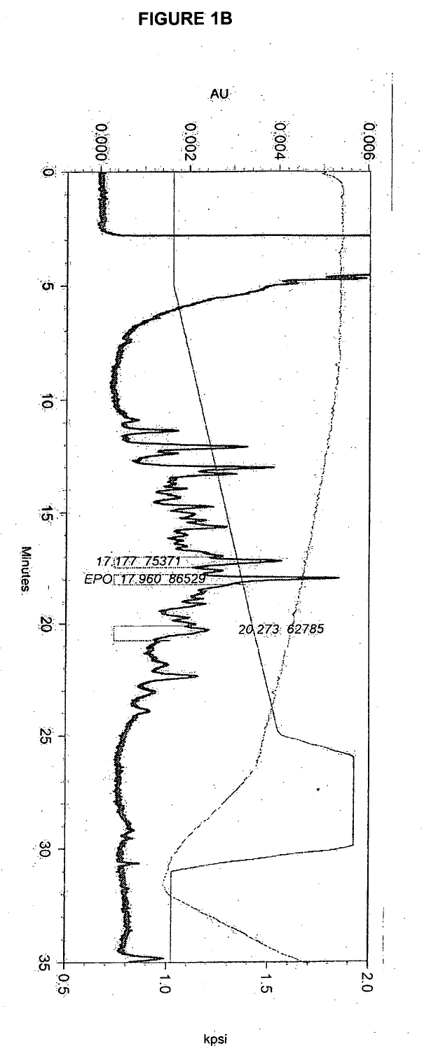

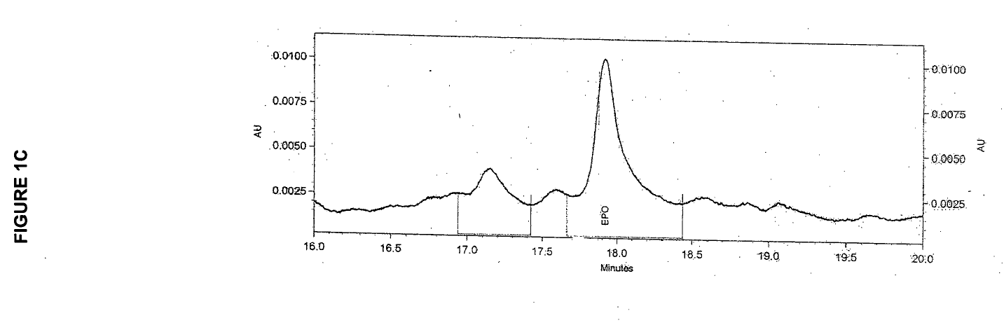

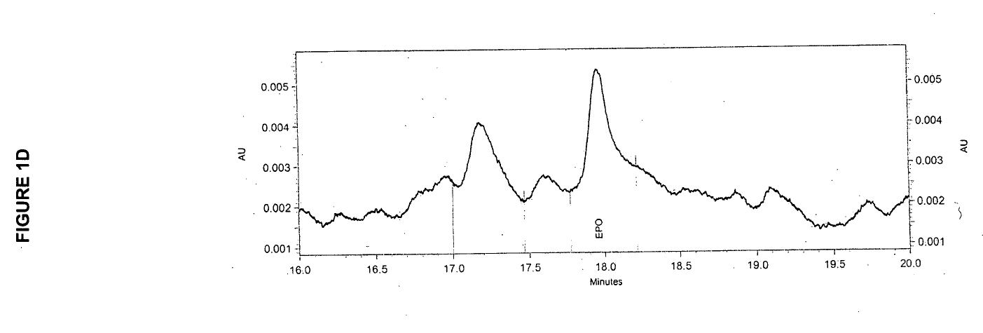

The data in Table 8 below show that the addition of lipid mixture to the cell culture helps to maintains cell viability, regardless of the viability at infection. The three runs with lipid addition had an average drop in viability of 4%, significantly lower than the 15% drop in the run with no lipid addition. Also, based on the runs with similar MOI's, the more lipid mix added, the higher the EPO titer. The run with 1.5% v/v lipid addition produced an EPO titer that was 82% higher than the control run with no lipid addition and 35% higher than the run with 1.0% v/v supplementation. In addition to improving EPO titers, the lipid mixture has a dramatic effect on EPO quality, as seen in the RP-HPLC profiles shown in FIG. 1.

TABLE 8
Effects of Fresh Lipid Supplementation

| | Infection VCD | *Harvest VCD | | EPO titer | |
|---|---|---|---|---|---|
| | (10⁶ cells/mL)/ | (10⁶ cells/mL)/ | | RP-HPLC | Lipid |
| | Viability (%) | Viability (%) | MOI | (mg/L) | Mix |
| Experiment | 6/>90 | 7.22/89.4 | 0.8 | 18.3 | 1.0% |
| 1 | | | | | |
| Experiment | 4.02/74.3 | 6.31/74.2 | 0.2 | 34.8 | 1.5% |
| 2A | | | | | |
| Experiment | 5.04/90.7 | 9.42/83.0 | 0.1 | 25.7 | 1.0% |
| 2B | | | | | |
| Experiment | 3.54/81.4 | 6.95/69.2 | 0.2 | 19.1 | None |
| 2C | | | | | |

*harvest time = 65 hr post-infection

2.4 Study II: Fresh Lipid Addition vs Addition of Commercially Available Lipid Mix
Experiment 3

2 L Sf9 cells with VCD of 5.17×10⁶ cells/mL and viability of 97% were transferred to fermenter Z-1100E containing 5 L Sf-900II media. After 2 days, when VCD=2.9×10⁶ cells/mL and viability=91.9%, 150 mL of the commercially available lipid mix was added aseptically through the 19 mm head port/septum on Z-1100E to give a final lipid addition of 1% v/v. Cells were allowed to acclimate to the lipid mix for 1 hour. After 1 h, the cells were infected with 60 mL concentrated virus (titer=1.2×10⁸ pfu/mL as determined by the standard plaque assay). An additional 3 L of Sf-900II media was added after infection. The run parameters are outlined in the Table below.

TABLE 9
Summary of Parameters for Experiment 3

| Pre-Infection Parameters | |
|---|---|
| Agitation (rpm)/Impellor Type | 40/pitched blade (45°) |
| Aeration (lpm) | 0.3 |
| Temperature (° C.) | 27.0 |
| DO Setpoint (%) | 60 |
| Post-Infection Parameters | |
| Agitation (rpm) | 60 |
| Aeration (lpm) | 0.4 |
| Temperature (° C.) | 27.0 |
| DO Setpoint (%) | 60 |

Experiment 4

2 L Sf9 cells with VCD of 5.10×10⁶ cells/mL and viability of 97% were transferred to fermenter Z-1100F containing 5 L Sf-900II media. After 2 days, when VCD=2.2×10⁶ cells/mL and viability=89.1%, 150 mL fresh lipid mix (prepared according to the protocol in Example 1) was added aseptically through the 19 mm head port/septum on Z-1100F to give a final lipid addition of 1%. Cells were allowed to acclimate to the lipid mix for 1 hour. After 1 hr, the cells were infected with 55 mL concentrated virus (titer=1.2×10⁸ pfu/mL as determined by the standard plaque assay). An additional 3 L of Sf-900II media was added after infection. The run parameters are outlined in the Table 10 below.

TABLE 10
Summary of Parameters for Experiment 4

| Pre-Infection Parameters | |
|---|---|
| Agitation (rpm)/Impellor Type | 40/pitched blade (45°) |
| Aeration (lpm) | 0.3 |
| Temperature (° C.) | 27.0 |
| DO Setpoint (%) | 60 |
| Post-Infection Parameters | |
| Agitation (rpm) | 60 |
| Aeration (lpm) | 0.4 |
| Temperature (° C.) | 27.0 |
| DO Setpoint (%) | 60 |

The data in Table 11 below demonstrate that when fresh lipid is added at the time of infection the culture produces significantly higher EPO titers, than a similar culture supplemented with a commercial lipid mixture. In this experiment, the difference in EPO titers was about 38%.

TABLE 11
Fresh Lipid Mixture vs. Commercial Lipid Mixture

| | Infection VCD | *Harvest VCD | EPO titer | EPO titer | |
|---|---|---|---|---|---|
| | (10⁶ cells/mL) | (10⁶ cells/mL) | RP-HPLC | ELISA | Type of Lipid |
| | Viability (%) | Viability (%) | (mg/L) | (mg/L) | Mixture |
| Experiment 3 | 2.9/91.9 | 4.69/85.9 | 14.5 | 13.2 | Commercial |
| Experiment 4 | 2.17/89.1 | 5.73/86.8 | 20.1 | 21.4 | Fresh |

*harvest time = 66 hr post-infection

2.5 Conclusions Drawn from Experiments 1 to 4
1. A 1.5% (of total volume) supplementation of lipids, either freshly prepared lipids or the commercially available chemically defined lipid mix, improves the amount of completely glycosylated EPO produced.
2. Freshly prepared lipid mix outperforms commercially available chemically defined lipids by 38% based on EPO titer.
3. Fresh lipid mix and commercially available chemically defined lipid mix help the cells maintain higher viability through 65 hours of infection compared to runs with no lipid supplementation.
4. A 1.5% (v/v) supplementation of fresh lipid mix yields about 35% higher EPO titers than addition of 1% (v/v) lipid mix and about 82% higher EPO titers than no lipid addition.
5. A 1.5% (of total volume) supplementation of lipids, either freshly prepared lipids or the commercially available chemically defined lipid mix, improves cell culture broth quality by decreasing the ratio of impurities to EPO.

### Example 3

### Production of EPO Peptide at 100 L Scale in Insect Cell Lines

Erythropoietin (EPO) is an extra-cellular protein expressed in the BEV system. Insect cell culture development work was initiated to maximize the EPO titer yields in 20 L bioreactors. The reproducibility of EPO titer and culture broth quality in the 20 L bioreactors showed that >15 mg/L of EPO and clean culture broths were achievable, indicating that a successful insect cell culture process was developed for EPO production at that scale. The present series of bioreactor runs reproduce the EPO production process at the 100 L scale. Process consistency was also investigated at the 100 L scale. In order to evaluate the process stability (or consistency), the operating parameters were maintained the same as those found during the course of process optimization at the 20 L scale.

3.1 Materials

Allegheny Bradford. 100 L Fermenter
B. Braun Biostat C 20 L Fermenter
Coming Corp. Fembach Disposable 3 L Flask
GMP Sf-9 working cell bank
GMP baculovirus stock solution
B Braun Certomat BS 1 Shaking Incubator
Guava Technology Inc. Guava PCA System
Fisher Hamilton Class II Biological Safety Cabinet
Getinge Novus I Autoclave
   3.2. Experimental

The basal media used was Sf-900 II from Invitrogen Corporation. The cell line used in this work was Sf-9. A recombinant virus containing a piece of the gene encoding human red blood cell growth hormone erythropoetin (EPO) was used. Sf-9 cell density and viability used at the time of infection were ˜0.4-0.6×10⁷ cells/mL and ˜90%, respectively. The cell density and viability were measured using a Guava PCA System according. EPO concentration was determined by an ELISA method. The temperature was controlled at 27±0.50° C. and the agitation speed was set at 60 rpm for EPO production in 100 L fermenter. Yeastolate Ultrafiltrate was used to further increase cell densities to ˜0.8-1.2×10⁷ prior to viral infection. In order to obtain a consistent infection rate, cells were infected with a 0.2 MOI of concentrated stock virus. A freshly prepared lipid mix was added at a concentration of 1.5% into the culture medium an hour prior to viral infection. The dissolved oxygen level (DO level) of all runs was controlled at 60% to the end of the run.

Harvests were at 67 hours post infection. The components of lipid mix used for EPO production are as disclosed in Example 1 and exemplary process parameters for EPO production at the 100 L bioreactor scale are shown in the Table 12 below.

TABLE 12
Process Parameters for EPO Production in a 100L Bioreactor

| Component | 100L Bioreactot |
|---|---|
| Temperature | 27° C. |
| Agitation | 60 rpm |
| Impellers | Marine and BT6 |
| Aeration | 0.05 vvm |
| Dissolved Oxygen | 60% |
| MOI | 0.2 |

3.3 Experimental Results

The experimental results shown in Table 13 demonstrate that EPO production at 67 hours post infection was robust and reproducible. The calculated average EPO productivity in these 100 L runs was 22.7×3.9 mg/L. The average volumetric productivity for EPO production was 2270±330.9 mg. The average viable cell concentration at harvest for the two runs was 0.62±0.015×10⁷ cells/mL and the average cell viability at harvest was 86.3±0.25%. The IP/CE assay indicates that 72% and 100% of non-degraded EPO was found respectively, at harvest. These data demonstrate that the current process yields good quality EPO in titers greater than 15 mg/L.

TABLE 13
Summary of Several 100L EPO Production Runs

| | Batch No. | | |
|---|---|---|---|
| | 1 | 2 | Average |
| Number of Cell Passages | 18 | 18 | N/A |
| Initial Cell Density (1 × 10⁷ cells/mL)/ | 0.3 | 0.52 | 0.41 |
| Viability (%) | | | |
| Cell Density (1 × 10⁷ cells/mL)/ | 0.57/90 | 0.71/91 | 0.64/91.5 |
| Viability (%) (24 hour post infection) | | | |
| Cell Density (1 × 10⁷ cells/mL)/ | 0.6/86 | 0.63/86.5 | 0.62/86.3 |
| Viability (%) (67 hour post infection) | | | |
| Controlled Dissolved Oxygen (%) | 60 | 60 | 60 |
| Time Harvested (hours post infection) | 67.0 | 67.0 | 67 |
| EPO Concentration (mg/L) | 18.8 | 26.6 | 22.7 |
| Volumetric Productivity of EPO (mg) | 1880 | 2660 | 2270 |
| Non-Degraded (S0) EPO (%) | 72 | 100 | 86 |

### Example 4

### Production of EPO Peptide at a 1000 L Scale in Insect Cell Lines

This study evaluated the reproducibility of the insect cell culture process for producing EPO at the 1000 L scale.

4.1 Materials

Allegheny Bradford. 1000 L Fermenter
Allegheny Bradford. 150 L Fermenter
B. Braun Biostat C 20 L Fermenter
Coming Corp. Fembach Disposable 3 L Flask
GMP Sf-9 working cell bank
GMP baculovirus stock solution
B Braun Certomat BS1 Shaking Incubator
Guava Technology Inc. Guava PCA System
Fisher Hamilton Class II Biological Safety Cabinet
Getinge Novus I Autoclave
   4.2 Experimental

The basal media used in this study was Sf-900 II from Invitrogen Corporation. A recombinant virus containing a piece of the gene encoding human red blood cell growth hormone erythropoetin (EPO), and the Sf9 insect cell line were used in this work.

Sf9 insect cells were grown at 27° C. in shake flasks until they reached a cell density of 6×10⁶ and a viability of 98% at which point five liters of shake flask cells were added to a bioreactor containing 10 L of Sf-900II media. The final 15 L working volume was agitated at 50 rpm and aerated at a vvm of 0.04. When the cells again reached a density of 6×10⁶ (viability of 97%), the entire 15 L were transferred to the 100 L tank containing 35 L of Sf-900II media. At the time of cell transfer, 750 mL of fresh lipid mix was added to help stabilize the Sf9 cells. After one cell doubling, an additional 50 L of Sf-900II media was added. The tank was agitated at 50 rpm and aerated with a vvm of 0.05. When the cells again reached a cell density of 6×10⁶ (93% viability), the entire 100 L of SF 9 cells were transferred to the 1000 L tank containing 200 L of Sf-900II media and 4.5 L of fresh lipid mix. The tank was agitated at 20 rpm and aerated at a vvm of 0.083. When the cells reached a density of 12×10⁶ an additional 300 L of media was added followed by adding a second dose of fresh lipids (1.5%). After one hour, the cells were infected with an MOI of 0.2. The tank rpm was increased to 30 and the vvm was reduced to 0.6.

The temperature was controlled at 27±0.5° C. for all runs and the DO level (dissolved oxygen level) was controlled at 60%. Cell densities and viabilities were measured using a Guava PCA System. Harvest was at 67 h post infection. EPO concentration was determined by an ELISA method.

The components of lipid mix used for EPO production are as disclosed in Example 1 and the exemplary process parameters for EPO production at the 1000 L bioreactor scale are summarized in the Table 14 below.

TABLE 14
Process Parameters for EPO Production in a 1000 L Bioreactor

| Component | 1000 L Bioreactor |
|---|---|
| Temperature | 27 (C.) |
| Agitation | 30 rpm |
| Impellers | Marine and BT6 |
| Aeration | 0.06 vvm |
| Dissolved Oxygen | 60% |
| MOI | 0.2 |

4.3 Experimental Results

The experimental results shown Table 15 indicate that EPO production at 67 hours post infection was reproducible at the increased scale. The EPO titer based on the ELISA assay was 28.2 mg/L. The volumetric productivity for EPO production was 16920 mg. The viable cell concentration at harvest for the run was 0.75×10⁷ cells/mL and the cell viability at harvest was 90%. The IP/CE assay indicates that 100% of the EPO was non-degraded at harvest. These data demonstrate that the current process is scaleable to 1000 L and yields comparable quality EPO in titers greater than 25 mg/L.

TABLE 15
Parameter Summary for 1000 L EPO Production Run

| | |
|---|---|
| Number of Cell Passages | 18 |
| Initial Cell Density (1 × 10⁷ cells/mL)/ | 0.2/97 |
| Viability (%) | |
| Cell Density (1 × 10⁷ cells/mL)/ | 0.6/98 |
| Viability (%) (at infection) | |
| Cell Density (1 × 10⁷ cells/mL)/ | 0.75/90 |
| Viability (%) (67 hour post infection) | |
| Controlled Dissolved Oxygen (%) | 60 |
| Time Harvested (Hour post infection) | 67.0 |
| EPO Concentration (mg/L)-Elisa Assay | 28.2 |
| Volumetric Productivity of EPO (mg) | 16920 |
| Non-Degraded (S0) EPO (%) | 100 |

### Example 5

### EPO Purification using a 100 kDa and 10 kDa Tangential Flow Filtration (TFF) Cascade

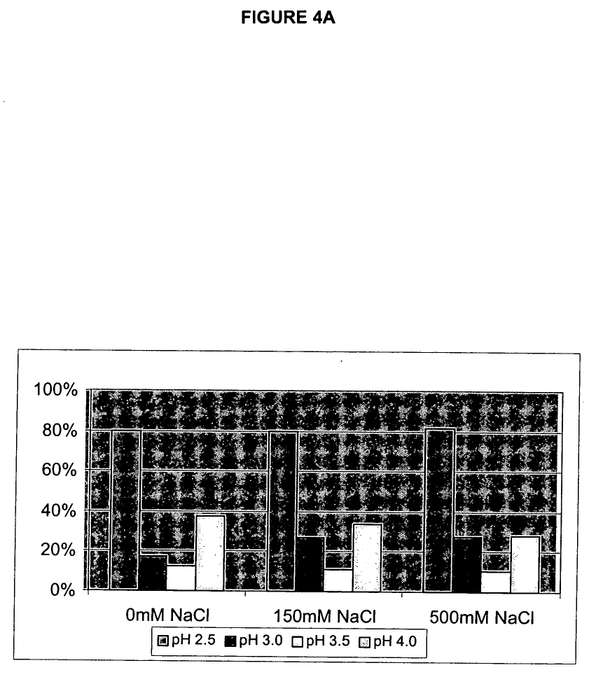

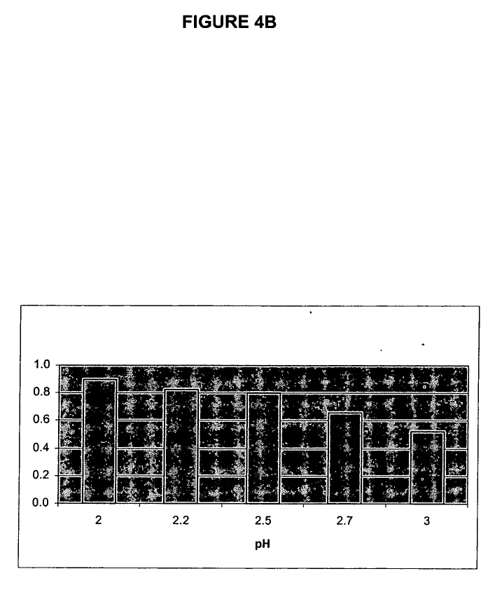

The following example illustrates the use of a cascade TFF membrane system for purification of peptides from insect cell culture. The TFF cascade is a two stage membrane separation system, which in this embodiment, uses two membranes with different pore sizes. As shown in the schematic diagram of FIG. 4, in the first stage, a 100 kDa Ultrafiltration (UF) membrane removes baculovirus, large proteins and DNA contaminants. The second stage, which employs a 10 kDa UF membrane, concentrates the permeate from the first stage. The EPO product is in the retentate from the second stage.

The described cascade TFF strategy significantly enriched the EPO protein, while it removes impurities including but not limited to baculovirus, large host proteins and host DNA. This strategy provides a new way of purifying recombinant proteins from baculovirus expression vector system (BEVS).

The TFF cascade membrane system with 100 kDa and 10 kDa membranes provides significant advantages for the peptide purification process. First, baculovirus and large protein impurities including the majority of glycosidases and large host DNA are removed from the product stream by the 100 kDa UF. Second, combining the filtration step with the addition of a protease/glycosidase inhibitor further improves EPO stability. The addition of enzyme inhibitors, makes it possible to operate the system at room temperature. Third, with the removal of contaminants by the 100 kDa membrane, the performance of the 10 kDa membrane is improved due to reduced loading. The higher membrane efficiency shortens the production time. More importantly, the load to the following chromatographic steps is much smaller due to the removal of impurity proteins. A smaller chromatography column is needed, and as a result overall production time is shorter and cost are lower.

Harvesting EPO From 600 L and 1000 L Cultures (from Example 4)

Cells from a 600 L culture were processed using a Alfa Laval Disc Stack Centrifuge for cell removal. To 10 L of the resulting supernatant was added sodium citrate to a final concentration of 5 mM. The pH of the solution was thus adjusted to 7.5. The 10 L centrate was then processed through a Millipore 10″ HC Opticap Plug-In Filter and subjected to a TFF cascade. The TFF cascade concentrated the initial 10 L to a 1 L volume and diafiltered the solution with 6 diavolumes of 20 mM HEPES 5 mM NaCitrate pH 7.5, across cascading Millipore 100 kDa and 10 kDa RC 1 sqft membranes. The resulting product was subjected to Mustang Q chromatography.

The 1 L was processed through a PALL Mustang Q AEX filter. The filter was washed with 20 mM HEPES 5 mM NaCitrate pH 7.5 to elute the peptide. The solution was stored at −20° C. for one week. The product was then subjected to Unosphere S chromatography. 900 mL Mustang Q flow through wash was loaded onto a 75 mL UNOSphere S Column. EPO was eluted with 5 column volumes (CV) of a 0.15 M NaCl stepwise gradient.

The EPO peptide quality in the resulting elution pool derived from this 600 L batch is comparable to the EPO quality obtained when processing a 1 L batch of cell culture. A representative western blot showing the quality of an EPO sample prepared and isolated according to the method disclosed in this example is shown in FIG. 5. The Figure also illustrates the uniform insect specific glycosylation pattern. The EPO recovery was 78% as determined by reverse phase HPLC. The purity of the EPO peptide in the elution pool was 92%, as determined by reverse phase HPLC.

### Example 6

### Purification Process for Human Erythropoietin (EPO)

The following Example illustrates a scalable purification process for human Erythropoietin (EPO). This peptide was expressed in the baculovirus expression vector system (BEVS) according to the method of the invention. EPO purified by this process can be used for the subsequent production of GlycoPEGylated EPO peptides.

The process includes cell removal by centrifugation, further clarification by depth filtration coupled with a 0.22 μm membrane filter, concentration and diafiltration using a tangential flow filtration cascade (TFF cascade), anion exchange membrane separation, viral inactivation by a non-ionic surfactant, cation exchange chromatography, viral inactivation by low pH, desalting by size exclusion chromatography, hydroxyapatite (HA) chromatography, hydrophobic interaction chromatography (HIC) and a final buffer exchange using ultrafiltration. The EPO purity from this process is sufficient to provide clinical trial quality peptide. The purity of the peptide was determined by SDS-PAGE gel electrophoresis, RP— and SEC-HPLC, as well as n-glycal analysis. Overall process yields (as determined by RP-HPLC) were 10-20% starting from cell culture at 15 L to 100 L scales.

6.1 Materials and Equipment

Centrifuge: LAPX-404 (Alfa Laval)
Depth filter: 90 SP (Cuno); alternatively 30 or 60 SP
Membrane filter: Millipak 0.22 μm (Millipore)
UF/DF membranes: Pellicon 2100 kDa/10 kDa for cascade, 5 kDa for final buffer exchange (Millipore)
Chromatography Resin: SP Sepharose FF cation exchange resin (GE Healthcare); SP
Sepharose HP cation exchange resin (GE Healthcare); UNOSphere S cation exchange
resin (Bio-Rad); G-25 resin size exclusion resin (GE Healthcare); HA type 1 resin (Bio-Rad); Butyl-4 FF hydrophobic interaction resin (GE Healthcare); Phenyl650 hydrophobic interaction resin (TosoHass).
Chromatography system: AKTA systems with Unicorn 5.01 software
SDS-PAGE was performed using 18% Tris-Glycine Gels and See Blue 2 molecular standards with colloidal blue and silver staining.
Buffer Systems: All buffers used will be described in the results section.
Cell culture: Cell culture was performed in 15 L and 100 L bio reactors.
   6.2 Experimental Procedure

The EPO was grown in cell culture using a baculovirus expression vector system. The Sf9 cells and cell debris were first removed from the cell culture by continuous centrifugation to bring the turbidity to <30 NTU. The supernatant was filtered through a depth filter/membrane filter train to bring the turbidity to <5 NTU. The resulting material was passed through a 100 kDa TFF membrane. The permeate from this first ultrafiltration step was passed through a 10 kDa TFF membrane. The retentate from this second ultrafiltration step was collected. This TFF cascade procedure was used to condition the mixture for subsequent purification steps by concentrating the mixture and affect a buffer exchange.

The resulting product was subsequently loaded onto a pre-equilibrated Mustang Q anion exchange membrane filter and the flow-through as well as the wash pool were collected. Triton X-100 (0.05% v/v) was then added and the mixture was held overnight at 4° C. before it was loaded onto a cation exchange column. The peptide containing fractions (gel analysis) were pooled and the mixture was held overnight at 4° C.

A second viral inactivation step was performed by spiking the cation exchange column pool to 20 mM citric acid and adjusting the pH to 2.1±0.1 with HCL and then holding the mixture at this low pH for 1 hour before adjusting the pH back to pH 7.5 with NaOH.

The mixture was then desalted using a size exclusion column to bring the conductivity to <1 mS/cm. The desalted mixture was then loaded onto a hydroxyapatite (HA) column. The elution pool from the HA column was brought to 0.5M sodium sulfate 10 mM sodium acetate and adjusted to pH 5.0. The resulting intermediate was then processed over a hydrophobic interaction resin. The eluate pool from the HIC column was diafiltered across a 5 kDa TFF membrane and the retentate was reconstituted in storage buffer to reach an EPO peptide concentration of approximately 2 mg/mL.

6.2.1 Removal of Cell Debris by Centrifugation

Prior to harvest the cell culture was chilled to 4° C. in the bioreactor and samples were pulled to determine the percentage of solids in the cell culture fluid. Cell removal was performed using a LAPX-404 disk stack centrifuge at a bowl speed of 6000 rpm and a flow rate of 2 liters per minute. The discharge interval was determined by the percentage of solids and the flow rate and was adjusted to not exceed 80% of the bowl's capacity for solids. Before discharging, the cell culture fluid was flushed from the bowl with 20 mM HEPES 150 mM NaCl pH 7.5 at 4° C. The liquid supernatant was collected and the solids (pellet) were discarded.

6.2.2 Clarification of the Supernatant by Depth/Membrane Filter Train

The supernatant from the disk stack centrifugation step was clarified through a 90 SP grade depth filter at 62.5 L/m² and a flow rate of 120 LMH. The filtrate from the 90 SP step was passed through a 0.22 μm Millipak 200 filter membrane at 1000 L/m² and a flow rate of 1920 LMH. Once all the supernatant was pumped into the filter housing the filter train was flushed with 20 mM HEPES 150 mM NaCl pH 7.5 at 4° C. The filtrate pool volume was thus adjusted to measure not less then the supernatant pool volume before filtration.

6.2.3 Conditioning by a Ultrafiltration and Diafiltration

Before chromatography, the clarified cell culture fluid was conditioned by concentration and diafiltration via a tangential flow filtration (TFF) cascade. To reduce the nonspecific binding of peptide to the membrane, regenerated cellulose membranes were used. The clarified cell culture fluid was concentrated to 1/10^{th} of the fermentation volume over a 100 kDa molecular weight cut off membrane and diafiltered with 5 diavolumes of 20 mM HEPES 150 mM NaCl pH 7.5. The permeate from the 100 kDa membrane was concentrated to 1/20^{th} of the fermentation volume over a 10 kDa molecular weight cut off membrane and diafiltered with 6 diavolumes of 20 mM HEPES pH 7.5. The 100 kDa membrane removes baculovirus and high molecular weight contaminants such as >100 kDa proteins and aggregates, the 10 kDa membrane clears water and low molecular weight contaminates. These steps are run concurrently and at controlled room temperature (20-25° C.).

6.2.4 Removal of Impurities by Mustang Q Membrane Filtration

The 10 kDa retentate pool was loaded onto a pre-equilibrated Mustang Q capsule filter at 250 L of feed per L Mustang Q membrane volume and a flow rate of 20 membrane volumes per minute. The filter capsule was flushed with 10 membrane volumes of 20 mM HEPES pH 7.5. The target protein passes through the filter and is collected in the flow through and wash fractions. Many host cell proteins, DNA and other acidic impurities as well as baculovirus are retained by the Mustang Q membrane.

6.2.5 Viral Inactivation by Non-Ionic Surfactant

The Mustang Q filtrate pool was spiked with a 10% stock solution of Triton X-100 to a final concentration of 0.05% v/v. The mixture was held at 4° C. overnight. This step targets the inactivation of enveloped viruses.

6.2.6 Purification of EPO by Cation Exchange Chromatography

The anion exchange pool was then applied to cation exchange chromatography. This step serves as the primary purification step and removes previously added Triton X-100 from the product mixture. In this experiment UNOSphere S from Bio-Rad Laboratories was used as the cation exchange resin and was equilibrated to 20 mM HEPES pH 7.5. Mustang Q pool was loaded onto the resin targeting 10 absorption units (280 nm) per mL of resin. Unbound proteins were washed from the column with 5 column volumes (CV) of equilibration buffer, and the bound proteins were eluted using a stepwise NaCl gradient to 200 mM NaCl in 20 mM HEPES pH 7.5. The peak, which elutes from the column first represents host cell proteins. The remaining fractions were collected as product pool. The column was stripped with 1M NaCl in 20 mM HEPES pH 7.5. The cation exchange step provided separation of EPO peptide from many host cell proteins.

6.2.7 Viral Inactivation by Low pH

The product pool from the cation exchange column was then subjected to a low-pH hold step, which is targeted at the inactivation of non-enveloped viruses. Citric acid was added to the EPO containing mixture to reach a final concentration of 20 mM. The pH was then adjusted to pH 2.1±0.1 with HCl. The mixture was held at this low pH for 1 hour and the pH was then adjusted to pH 7.5 with NaOH. This step was performed at controlled room temperature.

6.2.8 Desalting by Size Exclusion Chromatography

Product pool was desalted in preparation for HA chromatography and was passed over a G-25 coarse bead size exclusion resin to effect a final salt conductivity below 1 mS/cm. This low conductivity is necessary to ensure satisfactory peptide holding capacity of the HA column. The resin was equilibrated to 20 mM HEPES pH 7.5 and the product pool was then loaded onto the column at 15-20% CV. The resin was washed with 20 mM HEPES pH 7.5 peptide collection was initiated as the absorbance at 280 nm increased and collection was stopped when the absorbance approached baseline and before the conductivity of the flow through reached 2 mS/cm. The step took multiple injections to complete.

6.2.9 Purification by HA Chromatography

The hydroxyapatite resin (type I, 80 μm) was first charged with 0.1 M sodium phosphate, pH 7.5 and equilibrated with 20 mM HEPES pH 7.5. The desalted pool was loaded onto the resin targeting 10 absorbance units (280 nm) per mL of resin. The resin was washed with 20 mM HEPES pH 7.5 to remove unbound components and the target protein was eluted with a 20 CV gradient to 1M NaCl in 20 mM HEPES 20 mM Glycine pH 7.5. The entire peak fractions were collected as product pool, and the column was stripped with 0.1M sodium phosphate pH 7.5. This step provides orthogonal purification and removes background host cell proteins, DNA, and endotoxin.

6.2.10 Purification by Hydrophobic Interaction Chromatograihy

To the HA eluate was added sodium sulfate (0.5M) in 10 mM sodium acetate and the pH was adjusted to pH 5.5. The Butyl-4 FF column was equilibrated to 0.5M sodium sulfate in 10 mM sodium acetate pH 5.5 and the product was then loaded onto the HIC column targeting 10 absorption (280 nm) units per mL of resin. Unbound impurities were washed from the column and the product was eluted with a step to 20 mM HEPES pH 7.5. The entire peak was collected as product and the column was stripped with 20% ethanol and high pH. This step provides polishing and specifically removal of a small-molecular weight contaminant (impurity A, FIG. 3).

6.2.11 Viral Removal by Membrane Filtration

The HIC eluate was filtered through a NFP viral removal filter. So far, this step was performed on a laboratory-scale but is intended to be used in at process-scale in the future.

6.2.12 Protein Concentration and Storage

The elution pool from the HIC column was concentrated to 2 mg/mL as determined by absorbance at 280 nm using a 5 kDa TFF membrane filter and was diafiltered with 3 diavolumes of 20 mM HEPES pH 7.5. The product was then stored at −20° C.

6.2.13 Process/Product Analysis

The process/product characterization was essentially based on SDS-PAGE gel analyses and RP-HPLC analyses.

### Example 7

### Viral Inactivation by a Low-pH Hold Procedure

This example describes a set of experiments investigating the effect of a low-pH hold step on EPO peptide recoveries. A low-pH hold step is useful as a viral kill step in the EPO purification process. A viral kill step is particularly important for the production of EPO used for clinical studies. The experiments investigated the effect of low pH on EPO peptide recovery while varying parameters such as pH, NaCl concentration, time and EPO concentration.

7.1 Materials

Citric Acid, Acetic Acid, HCL, NaOH, TRIS Base, Bulk EPO, UNOS pool EPO

7.2 Experimental

7.2.1 Investigating pH Range and NaCl Concentration

For the two sets of experiments investigating pH range and NaCl concentration, 10 mM Sodium Citrate 10 mM Sodium Acetate buffers were formulated to pH 2.0-4.0 and 0, 150, and 500 mM NaCl. Bulk EPO was then diluted ten fold in each formulation. Samples were held at room temperature for one hour, pH was then adjusted back to 7.5 with 1 M TRIS Base. Samples were analyzed by SDS-PAGE, RP-HPLC and SEC-HPLC. In a first experiment EPO samples were held at pH 2.5, 3.0, 3.5 and 4.0. In a second experiment further investigating the pH range between pH 2.0 and pH 3.0, samples were held at pH 2.0, 2.2, 2.5, 2.7 and 3.0.

Results for Experiment 1

On the SDS PAGE gel EPO bands are most intense in lanes corresponding to low pH holds at pH 2.5 and least intense in lanes corresponding to low pH hold at pH 3.5. No degradation or aggregation was observed in samples held at pH 2.5 regardless of NaCl concentration. RP-HPLC results show that, in this experiment, EPO peptide recovery is highest at pH 2.5 and is not related to ionic strength of the buffer as shown in FIG. 4A. SEC-HPLC results show no formation of aggregates in low pH hold samples and confirm highest EPO peptide recovery at pH 2.5 independent of NaCl concentration.

Results for Experiment 2

No agregation or degradation was detected on the SDS PAGE gel as a function of pH, and the intensity of the EPO bands appeared to be equivalent across the pH range tested (pH 2.0 to 3.0). However, RP-HPLC analysis of the samples indicated highest recovery at pH 2.0 and the data shows a trend of increasing EPO peptide recovery with decreasing pH as shown in FIG. 4B. SEC-HPLC analyses reveal no aggregate formation for any of the samples held between pH 2.0 and 2.5.

7.2.2 Investigating Time and EPO Peptide Concentration

For those experiments investigating time and EPO concentration, bulk EPO was adjusted to pH 2.3 with HCL. Samples were set aside for a time-course of 1 to 3.5 hours at room temperature. To investigate the effect of EPO concentration, EPO samples were diluted to desired EPO concentrations with 10 mM Sodium Citrate 10 mM Sodium Acetate pH 2.3. All samples were held for 1 hour at room temperature. The pH was then adjusted back to pH 7.5 with 1M TRIS base. Controls at pH 2.5 and pH 7.5 were diluted ten fold into buffer. Samples were analyzed by SDS-PAGE, RP-HPLC and SEC-HPLC.

Results

For the time course, no aggregation or degradation was detectable on the SDS PAGE gel. EPO intensity was equivalent across the time course samples from 0 to 3.5 hours. The observation was confirmed by RP-HPLC chromatography. HPLC chromatograms show equivalent EPO peptide recovery for low pH holds from 1-3.5 hrs and on EPO concentrations from 40-850 μg/mL.

7.2.3 Investigating the Effect of a Low-pH hold on the Isoelectric Point of EPO

360 mL of UNOS pool EPO was split in half. 180 mLs were brought to 20 mM Citric Acid with a 1M citric acid stock, adjusted to pH 2.2 with HCl, held at room temperature for 1 hour, then adjusted to pH 7.5 with 1M TRIS Base. Both halves of the UNOS pool were desalted over G-25 coarse bead, 900 cm/hr, and 16% CV injection. Further purification of the EPO was performed by chromatography on an HA column for both desalted pools. Samples were then analyzed by SDS-PAGE, IEF, RP-HPLC and SEC-HPLC.

Result

The IEF gel of the HA pool shows that the isoelectric point (pI) of EPO is unaltered by the low pH hold during the process. Therefore, the low pH hold may not cause deamidation or other degradations affecting the pl.

7.3 Conclusions

These data demonstrate that a low-pH hold a pH hold at about pH 2.0 to about 2.3 has only a minimal effect on EPO peptide stability, allowing a low pH hold to be incorporated into the EPO purification process. For maximum yield, the pH should be between about 2.0 and about 2.2. The pool can be held for at least 3.5 hours at room temperature (20-25° C.). The step can be performed on solutions with EPO concentrations between about 40 and about 850 μg/mL.

### Example 8

### Effects of Desalting the Loading Buffer on HA Column Capacity and The Effect of Glycine on EPO Peptide Recovery During HA Chromatography

In order to increase the robustness and scalability of the HA chromatography step for the production of clinical batches of EPO peptide, the effect of lowering the salt conductivity of the loading buffer on the loading capacity of the hydroxyapatite (HA) column was investigated. UNOsphere S pool containing EPO peptide was either diluted or desalted to decrease the conductivity. The product was then loaded onto an HA column at pH 7.5 and subsequently eluted using a buffer containing an appropriate concentration of sodium chloride.

8.1 Materials

HA type 1 resin 80 μm bead from BioRad
G-25 Coarse bead resin from GE Healthcare
XK style columns from GE Healthcare
Buffer: 0.1M Na₂PO₄ (Equilibration/Regeneration), 20 mM HEPES pH 7.5 (Equilibration/Wash/Elution), 20 mM HEPES 1 M NaCl pH 7.5 (Elution), 20 mM Glycine 1M NaCl pH 8.5 (Elution)
   8.2 Experimental

The HA load was conditioned by either dilution or desalting to reduce the conductivity of the load. An Äkta system was then used to load the feed stream onto a HA column (type 1 resin). The product was eluted with sodium chloride, and the column was stripped with sodium phosphate. The pH and the conductivity of the buffer during the chromatography were monitored and recorded by the Äkta system. The column load and resulting fractions were analyzed by SDS PAGE and RP-HPLC.

The UNOsphere S pool was desalted into 20 mM HEPES pH 7.5 over G-25 resin. Sample injection was 16-20% CV. The flow rate varied from 90-350 cm/hr with column hardware and system pressure constraints.

Experiment 1: Effect of different EPO Concentrations in the Load on HA recovery

TABLE 16
Experimental Parameters for Experiment 1

| | |
|---|---|
| Column dimensions | 0.66 cm × 6 cm |
| Column volume | 2.0 mL |
| Equilibration, elution, and | 5.0 mL/min = 877 cm/hr |
| strip flow rate | |
| Load flow rate | 5.1 mL/min = 894 cm/hr |
| Column residence time | 24 seconds |
| Mass loading | 4 mg/mL |
| Equilibration buffer | 20 mM HEPES pH 7.0 |
| Limit buffer | 20 mM Glycine 1M NaCl pH 9.2 |
| [EPO] in the load | 37.5-150 μg/mL |
| Load conditioning | Desalting |
| Conductivity in the load | 0.33 mS/cm |

Result

The load experiment tested EPO concentrations of 37.5-150 μg/mL. Equivalent purity and recovery was seen when the EPO concentration in the load was between 37.5 and 168 μg/mL. Results showed pool purity (100%) and recovery (90%) to be independent of the concentration of product in the load. Purity of 99% and recovery of 82% was seen with product concentrations as high as 168 μg/mL.

Experiment 2: Comparison of Desalted to Diluted Load

TABLE 17
Experimental Parameters for Experiment 2

| | |
|---|---|
| Column dimensions | 0.66 cm × 16 cm |
| Column volume | 5.5 mL |
| Equilibration, elution, and | 5.0 mL/min = 877 cm/hr |
| strip flow rate | |
| Load flow rate | 5.1 mL/min = 894 cm/hr |
| Column residence time | 64 seconds |
| Mass loading | 10 mg/mL |
| Equilibration buffer | 20 mM HEPES pH 7.0 |
| Limit buffer | 20 mM Glycine 1M NaCl pH 9.3 |
| EPO in the load | 97 μg/mL and 35.5 μg/mL |
| Load conditioning | Desalting and Dilution |
| Conductivity in the load | 0.44 mS/cm and 3.9 mS/cm |

Result

In this experiment the effect of either diluting or desalting the loading buffer on the recovery of EPO peptide during HA column chromatography was investigated. FIG. 5 indicates that 10% breakthrough is reached before loading 2 mg/mL with a diluted load, while this level of breakthrough is not achieved even after loading >9 mg/mL when the load is desalted. With a diluted load the step recovery is 45%, with the desalted load the step recovery is 74% if the pH 9 wash is excluded, and 90% if it is included. Both runs result in pool purity of >95 by RP-HPLC analysis.

Experiment 3: Loading Capacity of HA Chromatography Resin with Desalted Load

TABLE 18
Experimental Parameters for Experiment 3

| | |
|---|---|
| Column dimensions | 0.66 cm × 10 cm |
| Column volume | 3.4 mL |
| Equilibration, elution, | 5.0 mL/min = 877 cm/hr |
| and strip flow rate | |
| Load flow rate | 5.1 mL/min = 894 cm/hr |
| Column residence time | 40 seconds |
| Mass loading | 10 mg/mL |
| Equilibration buffer | 20 mM HEPES pH 7.0 |
| Limit buffer | 20 mM Glycine 1M NaCl pH 9.2 |
| EPO concentration in the load | 75 μg/mL |
| Load conditioning | Desalting |
| Conductivity in the load | 0.33 mS/cm |

Result

Experiment 3 was performed by desalting the load and testing the capacity of the column for EPO loadings of up to 10 mg/mL. Results show the capacity of the HA column to be greater than 10 mg/mL when the conductivity of the load is ≦0.33 mS/cm.

Experiment 4: Effect of 20 mM Glycine in Elution Buffer on Peptide Recovery

TABLE 19
Experimental Parameters for Experiment 4

| | |
|---|---|
| Column dimensions | 1.0 cm × 14.5 cm |
| Column volume | 11.4 mL |
| Equilibration, elution, and | 12 mL/min = 917 cm/hr |
| strip flow rate | |
| Load flow rate | 12 mL/min = 917 cm/hr |
| Column residence time | 57 seconds |
| Mass loading | 8 mg/mL |
| Equilibration buffer | 20 mM HEPES pH 7.5 |
| Limit buffer | 20 mM HEPES 1M NaCl pH |
| | 7.5 ± 20 mM Glycine |
| [EPO] in the load | 168 μg/mL |
| Load conditioning | Desalting |
| Conductivity in the load | 1.2 mS/cm |

Result

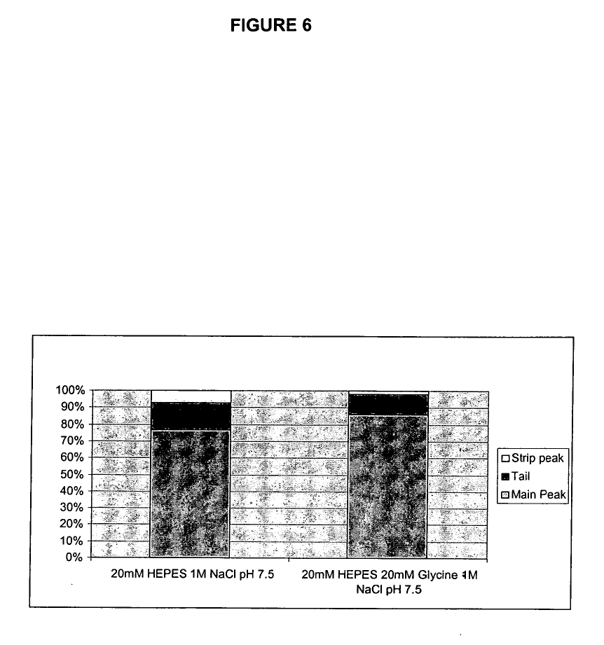

In this experiment the effect of 20 mM Glycine in the elution buffer at pH 7.5 on EPO peptide recovery during HA chromatography was evaluated. The addition of 20 mM Glycine to the elution buffer resulted in a sharper peak and increased step recovery by 5% as shown in FIG. 6. In addition, the addition of glycine allows for a lower than pH 9 elution buffer (e.g. pH 7.5 to 8.0) while essentially maintaining step recovery.

8.3 Conclusions Drawn from Experiments 1 to 4

1. HA column capacity increases with decreasing conductivity in the load. Conductivity of the HA load should be ≦1 mS/cm.
2. The addition of 20 mM glycine to the elution buffer increases the HA step recovery without decreasing pool purity. HA elution should be run at pH 7.5-8.0

### Example 9

### Purification Process for Chicken and Human ST6GalNAc I

The following Example illustrates a scalable purification process for both chicken and human Neu5Ac:GalNAc α 2,6-sialyltransferase (ST6GalNac I). These enzymes were expressed in the baculovirus expression vector system (BEVS) according to the methods described herein. ST6GalNAc I purified by this process can be used for the subsequent production of GlycoPEGylated peptides, e.g., G-CSF peptides or EPO peptides.

The process includes cell removal, CaCl₂ precipitation, concentration and diafiltration with UF/DF, Mustang Q membrane separation, UNOSphere S and hydroxyapatite chromatography, and a final buffer exchange using UF/DF. The ST6GalNAc I purity from this process is sufficient to produce TOX-ADME quality enzyme (estimated by SDS-PAGE). The overall process yield (as determined by enzyme activity) was 20-33% starting from cell culture at the 1 L scale.

9.1. Materials and Equipment

Chromatography Resin: Q and SP Sepharose XL (Q_{XL} and SP_{XL}) ion exchange resin (Amersham Biosciences); UNOSphere S cation exchange resin (Bio-Rad); HA ion exchange resin (Bio-Rad).
Chromatography Column: Omifit column (0.66 and 1.0 cm i.d.)
Chromatography system: AKTA purifier with Unicorn 5.01 software
Buffer filter: Nalgene 0.2 micron filter units (Nalgene)
SDS-PAGE was performed using 4-20% Tris-Glycine Gels and protein ladder molecular standards with Colloidal Blue Staining.
Buffer Systems:
All buffers used will be described in the results section.
Cell culture: Cell culture was performed in IL shake flasks.
   9.2. Experimental

SDS/PAGE with Coomassie Stain was used to identify ST6GalNAc I peptide. The product quality was determined by comparison of the Coomassie staining gel pattern with the standard peptide. Protein activity was measured by in vitro enzyme assay.

N-acetylgalactosaminide α 2-6-sialyltransferase I (GalNAc α 2-6-sialyltransferase I, ST6GalNAc I) is a member of the ST6GalNAc subfamily that exhibits activity toward GalNac-Ser/Thr, Galβ 1-3GalNAc-O-Ser-/Thr, and NeuAcα2-3Galβ1-3GalNAc-O-Ser/Thr. It exhibits type II membrane protein topology and has characteristic motifs for sialyltransferases called sialylmotifs L, S, and VS as well as the Kurosawa motif.

A small-scale purification process for the chicken variant of ST6GalNAc I expressed in sf9/BV cell culture includes cell removal, supernatant conditioning, SP Sepharose chromatography, hydrophobic interaction chromatography (HIC), and size exclusion chromatography (SEC).

In order to scale up this process, a large SP Sepharose column (10% of cell culture volume) would be needed for direct scaling. In addition, SP Sepharose co-purifies certain proteases and glycosidases, resulting in significant loss of activity during this step. Further, the preparation of HIC feed requires the addition of ammonium sulfate, which causes protein precipitation, resulting in ST6GalNAc I protein loss. In addition this step adds to the operational complexity and disposal costs. SEC is also not preferred in large-scale manufacturing due to low productivity, high production costs, and operational difficulty.

An alternative and innovative purification process was developed and is disclosed below. The developed process also provides guidance for the purification of other ST6 family enzymes and peptides in general.

The ST6GalNAc I was grown in cell culture using baculovirus expression vector system. The Sf9 cells and cell debris were first removed from the cell culture by batch centrifugation. The supernatant was treated with 10 mM CaCl₂ precipitation followed by a centrifugation clarification to remove the colloidal impurities and possibly baculovirus. The resulting material was conditioned by concentration and buffer exchange through a 10 kDa diafiltration membrane before loading onto a pre-equilibrated Mustang Q anion exchange membrane filter. The flow-through and wash pool were collected and used as the feed to a cation exchange column, UNOSphere S. The proper elution pool was collected based on gel and activity analyses, diluted 3-fold and used as the feed to a Hydroxyapatite (HA) column. The elution pool from this column was concentrated through a spin filter and diluted into an appropriate final storage buffer system.

9.2.1. Removal of Cell Debris by Centrifupation

Since ST6GalNAc I enzymes were extracellular proteins, the first step in the protein purification process was to remove Sf9 cells and cell debris. Considering that the cell culture volume was below 2 liters, batch centrifugation was used in our experiments. In the future, for a large-scale production, cell removal could be accomplished using filtration or continuous centrifugation at the end of cell culture operation. The obtained materials could be further clarified by an appropriate filter or filter train. At pilot scale (10-100 L), the cell and debris could be also removed directly by a proper filter train. For larger production scales, continuous centrifugation followed by filtration would be preferred.

Cell removal was monitored by the turbidity, which was measured by the absorbance of materials at 590 nm pre- and post-centrifugation. The centrifugation at different speeds for the same time, and at the same speed for different times does not make a big difference in the resulting turbidity of the supernatants.

The cell removal at different temperatures was also explored. As to the turbidity reduction, the same performance was observed in all the conditions. There was an indication that centrifugation efficiency heavily depended on the cell density and viability at the end of the cell culture. Lower viablility cultures at time of harvest correlated with greater impurity amounts in clarified cell culture fluid. The impurity proteins are likely released to the supernatant after lysis. A possible way to assess cell lysis would be to measure Lactate Dehydrogenase activity in culture supernatants as that is an intracellular protein that will only be detected upon cell lysis.

The cell culture supernatant containing ST6 was stored at −20° C. for 14 weeks before further processing.

9.2.2 CaCl₂ Precipitation

Calcium chloride was added to the supernatant and the pH of the solution was raised using base (e.g. NaOH). As a result, a precipitate forms that contains calcium hydroxide. This precipitation process can remove cell debris, colloidal impurities, and some baculovirus from the cell culture supernatant. The precipitated impurities can be removed by centrifugation at 5000 rpm. Additional Ca²⁺ is chelated by EDTA and further removed by subsequent UF/DF. The recovery of peptide in this step was high, ranging from 78-111% (enzyme activity assay).

9.2.3. Conditioning by Ultrafiltration and Diafiltration

Before chromatography, the CaCl₂ supernatant was conditioned by concentration and diafiltration via tangential flow filtration (TFF). To reduce the nonspecific binding of peptide to the membrane, a regenerated cellulose membrane with a 10 kDa molecular weight cut-off was used at all times. To save buffer and time, the supernatant from the CaCl₂ precipitation was first concentrated by 5× and then diafiltered with 5 diavolumes of the equilibration buffer used in the subsequent column chromatography step (all at room temperature, 20-25° C.).

To investigate the protease activity during the concentration and diafiltration process, direct diafiltration was performed as a control experiment. Cell culture material containing chicken ST6GalNAc I was used. A five-fold concentration by UF appears to cause a 50% loss of activity (mass recovery is not known). Without concentration, the protein activity was higher. This may indicate that degrading enzymes are enriched when cell culture fluid is concentrated by UF. A lower fold concentration e.g., 2×, 3× gives higher ST6GalNAc I activity recovery.

9.2.4 Purification by Ion Exchange Chromatography

Binding of Peptide to Q_{XL} resin at Different pH

A batch binding experiment was performed to determine at which pH chicken ST6GalNAc I expressed in BEVS binds to Q_{XL} resin. After CaCl₂ precipitation and clarification by centrifugation, the supernatant was concentrated 5-fold using a 10 kDa, 1 ft² membrane and diafiltered to 25 mM Na₂PO4/NaOAc, pH 7.5 with 5 buffer exchanges at room temperature. The ultrafiltration permeate flux was 50 LMH (liters/m²/hour). The retentate flow rate was 120 mL/min. The transmembrane pressure was 8 psi. The pH of retentate samples was adjusted to either pH 7.0, 6.5, 6.0, 5.5 or 5.0 with dilute acetic acid and to pH 8.0 and 8.5 with dilute NaOH. The obtained solutions were adsorbed to the corresponding pre-equilibrated Q_{XL} resin in a centrifugation tube. After washing with equilibration buffer, the peptide was eluted from the resins by the equilibration buffer containing 1.0 M NaCl. As the pH increased to 8.5, the ST6GalNac started to bind the resin. As the Q_{XL} column is intended to allow product to flow through, the pH of the elution buffer should be kept between 7.5 and 8.0.

Purification of ST6GalNAc I by Q_{XL}/SP_{XL} and Q_{XL}/SPHP Resins

The retentate from the concentration/diafiltration step was directly applied to a Q_{XL}/SP_{XL} column cascade. After washing with the equilibration buffer, the Q_{XL} column was disconnected and the peptides were eluted from the SP_{XL} resin using a linear gradient.

9.2.5. Purification by Mustang Q Membrane Filtration

In BEVS, protease activities are commonly observed and are frequently responsible for the degradation of expressed proteins. To maintain the protein integrity, protease inhibition is necessary. Alternatively, proteases can be removed as early as possible in the process. Mustang Q membrane filters are useful for this application. Similarly to Q_{XL}, the removal of unwanted proteins and host DNA could be achieved in this step. In addition, these filters are disposable, simple to install and use, and produce less pressure drop than the Q_{XL} column. Another advantage of this anion exchange filter is that, viruses could be cleared.

Both disk and capsule Mustang Q remove turbidity with a higher efficiency than Q_{XL} (as shown by AU at 590 nm). The total peptide recovery (as measured by UV 280 nm) is very similar for both Mustang Q and Q_{XL}. The majority of turbidity causing impurities was recovered in the Mustang Q wash. This was not the case for the Q_{XL} resin. Therefore, Q_{XL} is useful to irreversibly capture particulate contaminants.

Baculovirus Clearance by Mustang Q

The supernatant after cell removal was first diluted 3×, and the pH of the solution was adjusted to 7.5. This mixture was loaded onto a pre-equilibrated Mustang Q disk filter. Different filtrate pools were collected. The turbidity was determined by the absorbance at 590 nm while the virus titer in all pools was determined using a baculovirus plaque assay (Example 2).

In all collected filtrate pools, turbidity levels were very low. The baculovirus was removed by the filter, although it was not recovered in the elution from the filter. Therefore, Mustang Q can be used to remove virus particles and turbidity as well as other contaminants, such as unwanted proteins, and host DNA.

9.2.6. Purification by UNOSphere S

UNOSphere S from Bio-rad Laboratory was used to purify human and chicken ST6GalNAc I protein. Two peaks were observed in the elution process. The second peak was ST6GalNAc I. Thus, ST6GalNAc I protein can be further purified using this step. The second peak was collected as the product pool. No SDS-PAGE for different fractions was needed. The collected materials were used as the feed for subsequent column purification steps.

In one experiment the UNOSphere S column (7 mL, 570 CV) was loaded with 4 L cell culture containing human ST6GalNAc I. In another experiment the UNOSphere S column (7 mL, 280 CV) was loaded with 2 L culture containing chicken ST6GalNAc I. By concentrating and diafiltering cell culture fluid prior to chromatography, a much higher capacity was obtained compared with the 10 CV cell culture for SPFF column.

9.2.7. Purification by HA Chromatography

Hydroxyapatite (type I, 80 μm) was first charged with 0.4 M sodium phosphate, pH 6.8 and equilibrated with 5 mM sodium phosphate, 5 mM sodium sulfate, pH 7.5. The flow-through and chase pool from the Q_{XL} step containing human ST6GalNac I were loaded onto the HA beads. Then the beads were washed/eluted with 5 mM sodium phosphate, 5 mM sodium sulfate, pH 7.5, containing different concentrations of NaCl. The SDS-PAGE analysis of different samples showed that the major protein impurity, ecdysteroid UDP-glucosyltransferase (UGT) could be removed with a 0.3M NaCl wash (10 CV). ST6GalNAc I protein eluted at higher NaCl concentration. When the resin was regenerated with 0.4 M sodium phosphate, pH 6.8, no more ST6GalNAc eluted.

Chicken ST6GalNac I was also purified utilizing a HA column (type I, 40 or 80 μm). The column dimension was 0.66 id.×11.5 cm. The SP_{XL} pool was first buffer exchanged to the equilibration buffer used in the batch experiment and then applied to the HA column. The flow rate was 5 mL/min (876 cm/h). After a 10 CV wash at 0.3M NaCl, the protein was eluted using a linear gradient to 100% B in 20 CV. 100% B is equilibration buffer plus 1.0 M NaCl. A good separation between ST6GalNAc I and the impurity protein, UGT, was accomplished.

The elution gradient was optimized. After the 10 CV wash at 30% B, a linear gradient to 55% B in 15 and 20 CV was used to elute ST6GalNAc I from the HA column for human and chicken, respectively. Only the main peak was collected. The product obtained was of a high quality. A step gradient elution could also be used to obtain the purified product. The column loading for human ST6GalNAc I in the experiment was 4 L cell culture/4 mL HA (1 L/1 mL resin) while for chicken ST6GalNAc I was 2 L cell culture/4 mL HA (0.5 L/mL resin).

9.2.8. Protein Concentration and Storage

The elution pool from the HA column was concentrated using a ViaScience Spin filter 10 to 20 times and then formulated into 50 mM Bis-Tris, 0.1 M NaCl, pH 6.5 buffer. A UF/DF step can facilitate concentration of product from a large-scale production. The formulated product was stored at −20° C. in the formulation buffer buffer containing 50% (v/v) glycerol.

9.2.9 Process/Product Analysis

The process/product characterization was based on SDS-PAGE and enzyme activity analyses. The results showed that cell removal by centrifugation, and CaCl₂ precipitation are effective for removing major turbidity contamination including cells and other particulate contaminants. No protein separation was observed during UF/DF (save for small MW impurities cleared in the permeate), which is used for the feed preparation of following chromatography processes. Mustang Q removes not only the residual particle contaminants but also acidic impurities and virus particles. ST6GalNAc I can be further purified using UNOSphere S. Residual protein is further removed by HA column chromatography.

### Example 10

### The Purification of EPO from Baculovirus Expression Vector System (BEVS) Using Sartobind S

In this example, Sartorius Sartobind S, a cation exchange membrane filter, was used instead of the UNOSphere S column, employed in Example 6. Using this cation exchanger option, major protein impurities are removed. The Sartobind S material was tested in order to provide an alternative to UNOsphere S in this process application.

A typical flow through (FT) and wash (W) from a Mustang Q anion exchange column containing EPO peptide was used as the staring material for this experiment. This feed contained the typical impurities found in the EPO product at this stage of the purification process. The feed was applied to the Sartobind S membrane filter and the filter was washed. The majority of protein impurities present in the feed were removed in the flow-though and the chase as well as in the 1.0 M NaCl stripping. In addition, a small molecular weight impurity was removed in the 75 mM, 100 mM and 1000 mM NaCl elution steps. EPO peptide was eluted from the membrane adsorber by the elution buffer containing 50 mM NaCl. The resulting EPO peptide had a high purity (>90%). The described experiment was performed at least three times with very similar results.

In addition to providing EPO in high purity, Sartobind S has a number of additional advantages. Compared to traditional chromatography, Sartobind S is a disposal membrane filter. Therefore, no cleaning validation is needed. In addition, when using membrane adsorbers such as Sartobind S, a much higher flow rate can be used, relative to the flow rate typically used in column chromatography operations. As a result, higher purification productivity and efficiency can be achieved through Sartobind S. The faster and the more efficient the purification of protein proceeds, the smaller the chance that part of the protein is degraded by enzymes contained in the expression system. Thus, the overall manufacturing process for the production of peptides benefits from the incorporation of the Sartorius S cation exchange strategy. This alternative is especially useful for the purification of EPO.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of separating a peptide from an impurity with a molecular weight lower
than said peptide by hydrophobic interaction chromatography, said method comprising:
(a) applying a mixture comprising said peptide and said impurity to a hydrophobic interaction chromatography resin;
(b) eluting said impurity from said resin;
(c) eluting said peptide from said resin; and
(d) collecting an eluate fraction from (c) comprising said peptide, thereby separating said peptide from said impurity.

2. The method of claim 1, wherein at least about 50% of said impurity is removed by said method.

3. The method of claim 2, wherein at least about 90% of said impurity is removed by said method.

4. The method of claim 1, further comprising, prior to step (a):
(e) desalting a mixture comprising said peptide and said impurity, forming a desalted peptide mixture;
(f) eluting said desalted peptide mixture of step (e) from a hydroxyapatite chromatography medium; and
(g) collecting an eluate fraction from (f), comprising said peptide.

5. The method of claim 1, wherein said peptide comprises a substantially uniform insect-specific glycosylation pattern.

6. The method of claim 1, wherein said peptide is a member selected from erythropoietin, granulocyte colony stimulating factor, GNT1, GalT1, ST3Gal3, CST2, sialidase, GalNAcT2, Core1GalT, ST6GalNAc1, ST3Gal1, and ST3Gal2.

7. The method of claim 1, wherein said mixture comprising said peptide is provided by a procedure comprising:
(h) infecting insect cells in an insect cell culture with a recombinant baculovirus comprising a nucleotide sequence encoding said peptide
wherein
(i) said cell culture medium is supplemented with a lipid mixture; and
(ii) said infecting occurs in the culture medium supplemented with said lipid mixture; and
(i) growing the infected insect cells of (h) to produce a culture liquid comprising said peptide encoded by said nucleic acid sequence
wherein said peptide comprises an insect-specific glycosylation pattern.

8. The method of claim 7, wherein said lipid mixture is supplemented into said insect cell culture at a percentage of total culture volume equivalent to between about 0.5% to about 3% v/v.

9. The method of claim 7, wherein said lipid mixture is added to supplement said insect cell culture from between about 0.5 hours to about 2.0 hours prior to said infecting.

10. The method of claim 7, wherein said infecting employs a multiplicity of infection between about 10⁻⁸ to about 1.0.

11. The method of claim 7, wherein said lipid mixture comprises: an alcohol, a surfactant, a sterol, a detergent, an anti-oxidant, and a lipid source.

12. The method of claim 7, further comprising:
(j) removing cellular debris from said culture liquid to produce a first mixture comprising said peptide;
(k) conditioning said first mixture (j) using a tangential flow filtration cascade;
(l) adjusting pH of conditioned mixture from (k), forming a pH adjusted mixture;
(m) eluting said pH adjusted mixture from (l) from an anion-exchange medium;
(n) collecting an eluate fraction from (m) comprising said peptide;
(o) eluting collected eluate fraction from (n) from a cation-exchange medium;
(p) collecting an eluate fraction from (o) comprising said peptide;
(q) subjecting said eluate fraction from (p) to a low-pH hold procedure, forming a viral-inactivated peptide solution; and
(r) concentrating said viral inactivated peptide solution.

13. The method of claim 12, wherein said removing of (j) is accomplished by a member selected from batch centrifugation, continuous centrifugation, filtration, and continuous centrifugation followed by filtration.

14. The method of claim 13, wherein said continuous centrifugation is accomplished using a disk-stack centrifuge.

15. The method of claim 12, wherein said concentrating of (r) is accomplished by ultrafiltration.

16. The method of claim 1, further comprising:
(s) isolating said peptide from (i);
(t) contacting isolated peptide from (s) with a glycosyltransferase and a modified glycosyl donor, comprising a glycosyl moiety which is a substrate for said glycosyltransferase, which is covalently linked to a modifying group, under conditions appropriate for the formation of a covalent bond between said glycosyl moiety of said glycosyl donor and said peptide, thereby producing a modified glycopeptide; and
(u) purifying said modified glycopeptide.

17. A method of separating a peptide from an impurity by hydroxyapatite chromatography, said method comprising:
(a) desalting a mixture comprising said peptide and said impurity, forming a desalted peptide mixture;
(b) applying said desalted peptide mixture from (a) to a hydroxyapatite resin;
(c) washing said hydroxyapatite resin, removing said impurity from said resin;
(d) eluting said peptide from said resin with an elution buffer; and
(e) collecting an eluate fraction from (d) comprising said peptide, thereby separating said peptide from said impurity.

18. The method of claim 17, wherein said desalted mixture has a conductivity between about 0.1 mS/cm and about 4.0 mS/cm.

19. The method of claim 17, wherein said elution buffer comprises an amino acid.

20. The method of claim 19, wherein said amino acid is glycine.

21. The method of claim 20, wherein said glycine is added to said elution buffer at a final concentration of about 5 mM to about 50 mM.

22. The method of claim 17, wherein said peptide comprises a substantially uniform insect-specific glycosylation pattern.

23. The method of claim 17, wherein said peptide is a member selected from erythropoietin, granulocyte colony stimulating factor, GNT1, GalT1, ST3Gal3, CST2, sialidase, GalNAcT2, Core1GalT, ST6GalNAc1, ST3Gal1, and ST3Gal2.

24. The method of claim 17, wherein said mixture comprising said peptide is provided by a procedure comprising:
(f) infecting insect cells in an insect cell culture with a recombinant baculovirus comprising a nucleotide sequence encoding said peptide
wherein
(i) said cell culture is supplemented with a lipid mixture; and
(ii) said infecting occurs in said cell culture supplemented with said lipid mixture; and
(g) growing the infected insect cells of step (f) to produce a culture liquid comprising said peptide encoded by said nucleic acid sequence
wherein said peptide comprises an insect-specific glycosylation pattern.

25. The method of claim 24, wherein said lipid mixture is supplemented into said insect cell culture at a percentage of total culture volume equivalent to between about 0.5% to about 3% v/v.

26. The method of claim 24, wherein said lipid mixture is added to supplement said insect cell culture from between about 0.5 hours to about 2.0 hours prior to infecting.

27. The method of claim 24, wherein said infecting employs a multiplicity of infection between about 10⁻⁸ to about 1.0.

28. The method of claim 24, wherein said lipid mixture comprises: an alcohol, a surfactant, a sterol, a detergent, an anti-oxidant, and a lipid source.

29. The method of claim 24, further comprising:
(h) removing cellular debris from said culture liquid to produce a first mixture comprising said peptide;
(i) conditioning said first mixture from (h) using a tangential flow filtration cascade;
(j) adjusting pH of conditioned mixture from (i), forming a pH adjusted peptide mixture;
(k) eluting said pH adjusted mixture from 0) from an anion-exchange medium;
(l) collecting an eluate fraction from (k) comprising said peptide;
(m) eluting collected eluate fraction from (1) from a cation-exchange medium;
(n) collecting an eluate fraction from (m) comprising said peptide;
(o) subjecting collected eluate fraction from (n) to a low-pH hold procedure, forming a viral inactivated peptide mixture;
(p) eluting said viral inactivated mixture comprising from a hydrophobic interaction chromatography medium;
(q) collecting an eluate fraction comprising said peptide from (p); and
(r) concentrating said eluate fraction from (q).

30. The method of claim 29, wherein said removing of step (h) is accomplished by a member selected from batch centrifugation, continuous centrifugation, filtration, and continuous centrifugation followed by filtration.

31. The method of claim 30, wherein said continuous centrifugation is accomplished using a disk-stack centrifuge.

32. The method of claim 29, wherein said concentrating of step (r) is accomplished by ultrafiltration.

33. The method of claim 17, further comprising:
(s) isolating said peptide from (g);
(t) contacting isolated peptide from (s) with a glycosyltransferase and a modified glycosyl donor, comprising a glycosyl moiety which is a substrate for said glycosyltransferase, which is covalently linked to a modifying group, under conditions appropriate for the formation of a covalent bond between said glycosyl moiety of said glycosyl donor and said peptide, thereby producing a modified glycopeptide; and
(u) purifying said modified glycopeptide.

34. A method of preparing a viral inactivated peptide mixture by a low-pH hold procedure, said method comprising:
(a) lowering pH of a mixture comprising said peptide;
(b) maintaining said pH of step (a) for a selected period of time; and
(c) raising said pH of said mixture comprising said peptide, forming a viral-inactivated peptide mixture.

35. The method of claim 34, wherein said pH of step (a) is lowered to between about pH 2.0 and about pH 4.0.

36. The method of claim 35, wherein said pH of step (a) is lowered to between about pH 2.0 and about pH 2.5.

37. The method of claim 34, wherein said period of time is selected from between about 30 minutes and about 2 hours.

38. The method of claim 37, wherein said period of time is about 1 hour.

39. The method of claim 34, wherein said peptide comprises a substantially uniform insect-specific glycosylation pattern.

40. The method of claim 34, wherein said peptide is a member selected from erythropoietin, granulocyte colony stimulating factor, GNT1, GalT1, ST3Gal3, CST2, Sialidase, GalNAcT2, Core1GalT, ST6GalNAc1, ST3Gal1, and ST3Gal2.

41. The method of claim 34, wherein said mixture comprising said peptide is provided by a procedure comprising:
(d) infecting insect cells in an insect cell culture with a recombinant baculovirus comprising a nucleotide sequence encoding said peptide
wherein
(i) said cell culture is supplemented with a lipid mixture; and
(ii) said infecting occurs in said cell culture supplemented with said lipid mixture; and
(e) growing the infected insect cells of step (d) to produce a culture liquid comprising said peptide encoded by said nucleic acid sequence
wherein said peptide comprises an insect-specific glycosylation pattern.

42. The method of claim 41, wherein said lipid mixture is supplemented into said insect cell culture at a percentage of total culture volume equivalent to between about 0.5% to about 3% v/v.

43. The method of claim 41, wherein said lipid mixture is added to supplement said insect cell culture from between about 0.5 hours to about 2.0 hours prior to said infecting.

44. The method of claim 41, wherein said infecting employs a multiplicity of infection between about 10⁻⁸ to about 1.0.

45. The method of claim 41, wherein said lipid mixture comprises: an alcohol, a surfactant, a sterol, a detergent, an anti-oxidant, and a lipid source.

46. The method of claim 34, further comprising prior to (a):
(f) removing cellular and other debris from said insect cell culture to produce a first mixture comprising said peptide;
(g) conditioning said first mixture of step (f) using a tangential flow filtration cascade;
(h) adjusting pH of said conditioned mixture of step (g), forming a pH adjusted mixture;
(i) eluting pH adjusted mixture (h) from an anion-exchanger;
(j) collecting an eluate fraction from (i) comprising said peptide;
(k) eluting said eluate fraction from 0) from a cation-exchange medium; and
(l) collecting an eluate fraction from (k) comprising said peptide.

47. The method of claim 34, further comprising following (c):
(m) desalting said viral-inactivated peptide mixture of (c), forming a desalted peptide mixture
(n) eluting said desalted peptide mixture from (m) from a hydroxyapatite chromatography medium;
(o) collecting an eluate fraction from (n) comprising said peptide;
(p) subjecting said eluate fraction from (o) to hydrophobic interaction chromatography;
(q) collecting an eluate fraction from (p) comprising said peptide; and
(r) concentrating said eluate fraction from (q) comprising said peptide.

48. The method of claim 46, wherein said removing of step (f) is accomplished by a procedure, which is a member selected from batch centrifugation, continuous centrifugation, filtration, and continuous centrifugation followed by filtration.

49. The method of claim 46, wherein said continuous centrifugation is accomplished using a disk-stack centrifuge.

50. The method of claim 47, wherein said concentrating of step (r) is accomplished by ultrafiltration.

51. The method of claim 34, further comprising:
(s) isolating said peptide from (e);
(t) contacting isolated peptide from (s) with a glycosyltransferase and a modified glycosyl donor, comprising a glycosyl moiety which is a substrate for said glycosyltransferase, which is covalently linked to a modifying group, under conditions appropriate for the formation of a covalent bond between said glycosyl moiety of said glycosyl donor and said peptide, thereby producing a modified glycopeptide; and
(u) purifying said modified glycopeptide.

52. A method of purifying a peptide, said method comprising:
(a) conditioning a mixture comprising said peptide using a tangential flow filtration cascade wherein said conditioning occurs prior to subjecting said mixture to chromatographic purification steps.

53. The method of claim 52, wherein said conditioning comprises:
(i) ultrafiltering said mixture across a first ultrafiltration membrane;
(ii) ultrafiltering permeate from step (i) across a second ultrafiltration membrane; and
(iii) collecting retentate from step (ii).

54. The method of claim 53, wherein said first ultrafiltration membrane has a molecular weight cutoff of between about 50 kDa and about 150 kDa.

55. The method of claim 54, wherein said first ultrafiltration membrane has a molecular weight cutoff of about 100 kDa.

56. The method of claim 53, wherein said second ultrafiltration membrane has a molecular weight cutoff of between about 5 kDa and about 15 kDa.

57. The method of claim 56, wherein said second ultrafiltration membrane has a molecular weigh cutoff of about 10 kDa.

58. The method of claim 52, wherein said peptide comprises a substantially uniform insect-specific glycosylation pattern.

59. The method of claim 52, wherein said peptide is a member selected from erythropoietin, granulocyte colony stimulating factor, GNT1, GalT1, ST3Gal3, CST2, sialidase, GalNAcT2, Core1GalT, ST6GalNAc1, ST3Gal1, and ST3Gal2.

60. The method of claim 52, wherein said mixture comprising said peptide is provided by a procedure comprising:
(b) infecting insect cells in an insect cell culture with a recombinant baculovirus that comprises a nucleotide sequence encoding said peptide
wherein
(i) said cell culture is supplemented with a lipid mixture; and
(ii) said infecting occurs in the culture supplemented with said lipid mixture; and
(c) growing the infected insect cells of step (a) to produce a culture liquid comprising said peptide encoded by said nucleic acid sequence
wherein
said peptide comprises an insect-specific glycosylation pattern.

61. The method of claim 60, wherein said lipid mixture is supplemented into the insect cell culture at a percentage of the total culture volume equivalent to between about 0.5% to about 3% v/v.

62. The method of claim 60, wherein said lipid mixture is added to supplement the insect cell culture from between about 0.5 hours to about 2.0 hours prior to said infecting.

63. The method of claim 60, wherein said infecting employs a multiplicity of infection between about 10⁻⁸ to about 1.0.

64. The method of claim 60, wherein the lipid mixture comprises: an alcohol, a surfactant, a sterol, a detergent, an anti-oxidant, and a lipid source.

65. The method of claim 60, further comprising:
(d) removing cellular and other debris from said culture liquid to produce a first mixture comprising said peptide;
(e) adjusting pH of said first mixture comprising said peptide, forming a pH adjusted mixture;
(f) eluting said pH adjusted mixture comprising said peptide from (e) over an anion-exchanger;
(g) collecting an eluate fraction from (f) comprising said peptide;
(h) eluting said eluate fraction from (g) from a cation-exchange medium;
(i) collecting an eluate fraction from (h) comprising said peptide;
(j) subjecting said eluate fraction from (i) to a low-pH hold procedure, forming a viral inactivated peptide mixture;
(k) desalting said viral-inactivated peptide mixture from (j), forming a desalted peptide mixture;
(l) eluting said desalted peptide mixture of (k) from a hydroxyapatite chromatography medium;
(m) collecting an eluate fraction from (l), comprising said peptide;
(n) subjecting said eluate fraction from (m) to hydrophobic interaction 1 9 chromatography;
(o) collecting an eluate fraction from (n), comprising said peptide; and
(p) concentrating said eluate fraction.

66. The method of claim 65, wherein said removing of step (d) is accomplished by a member selected from batch centrifugation, continuous centrifugation, filtration, and continuous centrifugation followed by filtration.

67. The method of claim 66, wherein said continuous centrifugation is accomplished using a disk-stack centrifuge.

68. The method of claim 65, wherein said concentrating of step (p) is accomplished 2 by ultrafiltration.

69. The method of claim 52, further comprising:
(q) isolating said peptide from (c);
(r) contacting isolated peptide from (q) with a glycosyltransferase and a modified glycosyl donor, comprising a glycosyl moiety, which is a substrate for said glycosyltransferase, which is covalently linked to a modifying group, under conditions appropriate for the formation of a covalent bond between said glycosyl moiety of said glycosyl donor and said peptide, thereby producing a modified glycopeptide; and
(s) purifying said modified glycopeptide.

70. A method of purifying a peptide, said method comprising:
(a) removing cellular and other debris from a cell culture comprising said peptide, to produce a first mixture comprising said peptide;
(b) conditioning said first mixture of step (a) using a tangential flow filtration cascade, forming a conditioned mixture;
(c) adjusting pH of said conditioned mixture of step (b), forming a pH adjusted mixture;
(d) eluting said pH-adjusted conditioned mixture from step (c) from an an anion-exchange medium;
(e) collecting an eluate fraction from (d) comprising said peptide;
(f) eluting said eluate fraction from (e) from a cation exchange medium;
(g) collecting an eluate fraction from (f) comprising said peptide;
(h) subjecting said eluate fraction of (g) to a low-pH hold procedure producing a viral inactivated mixture comprising said peptide;
(i) desalting said viral inactivated mixture of step (h), forming a desalted mixture;
(j) eluting said desalted mixture of step (i) from a hydroxyapatite chromatography medium;
(k) collecting an eluate fraction comprising said peptide from (j);
(l) subjecting the eluate fractions of step (k) to hydrophobic interaction chromatography;
(m) collecting an eluate fraction from (l) comprising said peptide; and
(n) concentrating said eluate fraction from (m).

71. A lipid composition for use in conjunction with a baculovirus expression vector system, the composition comprising: an alcohol, a surfactant, a sterol, a detergent, an anti-oxidant, and a lipid source.

72. The composition of claim 71, comprising:
said alcohol in an amount between about 5% v/v to about 20% v/v;
said surfactant in an amount between about 5% w/v and about 15% w/v;
said sterol in an amount between about 0.02% to about 0.06% w/v;
said detergent in an amount between about 0.1% w/v to about 0.3% w/v,;
said anti-oxidant in an amount between about 0.01% w/v to about 0.05% w/v; and
said lipid source in an amount between about 0.05% w/v to about 0.25% w/v.
